(19) United States
(12) Patent Application Publication
(10) Pub. No.: US 2018/0256646 A1
(43) Pub. Date: Sep. 13, 2018

(54) COMPOSITIONS AND METHODS FOR MODULATING TOLL LIKE RECEPTOR SIGNAL

(71) Applicant: CASE WESTERN RESERVE UNIVERSITY, Cleveland, OH (US)

(72) Inventors: M. Edward Medof, Cleveland, OH (US); Jinbo Liu, Cleveland, OH (US); Michael G. Strainic, Cleveland, OH (US)

(21) Appl. No.: 15/974,307

(22) Filed: May 8, 2018

Related U.S. Application Data
(63) Continuation-in-part of application No. 15/950,038, filed on Apr. 10, 2018.
Application No. 15/950,038 is a continuation of application No. 15/077,256, filed on Mar. 22, 2016, now Pat. No. 9,937,206.
Application No. 15/077,256 is a continuation of application No. 13/505,976, filed on May 3, 2012, now Pat. No. 9,290,736.
Application No. 13/505,976 is a continuation-in-part of application No. 61/258,058, filed on Nov. 4, 2009.
Application No. 61/258,058 is a continuation-in-part of application No. 15/226,652, filed on Aug. 2, 2016.
Application No. 15/226,652 is a continuation of application No. 12/921,308, filed on Nov. 23, 2010, now abandoned.
(60) Provisional application No. 62/503,222, filed on May 8, 2017.
(60) Provisional application No. 61/034,303, filed on Mar. 6, 2008.

(51) Int. Cl. A61K 35/17; C12N 5/0783; A61K 38/00; A61K 35/12

(52) U.S. Cl. CPC A61K 35/17; C12N 5/0636; A61K 2035/124; A61K 2035/122; C12N 2501/998; A61K 38/00

(57) ABSTRACT

A method of method of inhibiting Toll like receptor (TLR) signaling in dendritic cells (DCs) of a subject in need thereof includes administering at least one complement antagonist to the DCs at an amount effective to substantially inhibits C3a receptor (C3aR) and/or C5a receptor (C5aR) signal transduction in the DCs induced by TLR signaling.

### RELATED APPLICATION

This application claims priority from U.S. Provisional Application No. 62/503,222, filed May 8, 2017, this application is also a Continuation-in-Part of U.S. Ser. No. 15/226,652, filed Aug. 2, 2016, which is a Continuation of U.S. patent application Ser. No. 12/921,308, filed Nov. 23, 2010, which is a National Phase Filing of PCT/US2009/036334, filed Mar. 6, 2009, which claims priority to U.S. Provisional Ser. No. 61/034,303, filed Mar. 6, 2009, this application is also a Continuation-in-Part of U.S. application Ser. No. 15/950,038, filed Apr. 10, 2018, which is a Continuation of U.S. Ser. No. 15/077,256, filed Mar. 22, 2016, (Now U.S. Pat. No. 9,937,206), which is a Continuation of U.S. Ser. No. 13/505,976, filed May 3, 2012, (Now U.S. Pat. No. 9,290,736), which is a National Phase Filing of PCT/US2010/055445, filed Nov. 4, 2010, which claims priority to U.S. Provisional No. 61/258,058, filed Nov. 4, 2009, the subject matter of which is incorporated herein by reference in its entirety.

### GOVERNMENT FUNDING

This invention was made with government support under Grant Nos. R01A35498 awarded by The National Institutes of Health (NIH). The United States government has certain rights to the invention.

### BACKGROUND

The evolutionarily and structurally conserved Toll-like receptors (TLRs) initiate innate immune activation which amplifies adaptive T and B cell immune responses. Both surface-expressed TLRs (e.g., TLRs 1, 2, 4-6, 11) and intracellularly localized TLRs recognize conserved molecular patterns derived from pathogens (i.e., pathogen associated molecular patterns or PAMPs) and potentially, some damaged/necrotic cells, e.g., HMGB1 which signals through TLR4. Upon ligation, TLRs initiate signaling cascades that while distinct, are transmitted through linker proteins that include “Myeloid Differentiation primary response gene 88” (MyD88) and “TIR-domain-containing adapter-inducing interferon-β” (TRIF, TICAM1) to activate gene expression profiles driven by transcription factors NF-κB, AP-1, and members of the interferon regulatory factor (IRF) family.

TLR sensing of PAMPs induces DC maturation. Important among processes associated with DC maturation are upregulation of MHC class II expression levels and induction of CD80, CD86 and CD40 costimulatory molecule expression as well as production of pro-inflammatory “innate” cytokines (e.g., type 1 interferon, IL-1, IL-6, IL-12, TNFα). Together, the TLR activation educates DCs to initiate appropriate effector T cell (T_{eff}) differentiation (e.g., Th1, Th2, Th17) and expansion, while limiting Foxp3⁺ T regulatory cell (T_{reg}) generation, function and stability. These combined effects provide protective, pathogen-reactive T cell immunity. As one example, signaling through TLR 3, TLR4, or TLR9, can cause DCs to elicit a Th1 response.

While these effects of TLR signaling protect the host from pathogens, non-physiological TLR activation can overcome normal immunological homeostasis and result in pathological immune injury. As examples, HMGB1/TLR4 activation is a crucial mediator of ischemia reperfusion injury and experimental CpG/TLR9 ligation can promote Th1 and Th17 cell activation in rodent models of autoimmunity. In transplant systems, TLR9-initiated signals overcome extended allograft survival induced by costimulatory blockade.

### SUMMARY

This application relates generally to a method of modulating Toll-like receptor (TLR) signaling in dendritic cells (DCs), and also to therapeutic methods of treating TLR, T cell, and/or B cell mediated disorders in a subject. It was found that ligation of TLR3, 4, and 9 induces DC production of complement components and local production of the anaphylatoxin C5a. In vitro, ex vivo, and in vivo analyses show that TLR-induced DC maturation and functional ability to stimulate T cell responses, requires autocrine C3a- and C5a-receptor (C3ar1/C5ar1) signaling. It was further found that TLR-initiated, DC autocrine C3ar1/C5ar1 signaling causes expansion of effector T cells and instability of regulatory T cells and contributes to T cell-dependent transplant rejection. Immune cell-derived complement production and autocrine/paracrine C3ar1/C5ar1 signaling are thus intermediary processes that link TLR-stimulation to DC maturation and the subsequent development of effector T cell responses.

Accordingly, in some embodiments, a method of treating a TLR, T cell, and/or B cell mediated disorder in a subject having or at risk of the TLR, T cell, and/or B cell mediated disorder includes administering to the subject an amount of at least one complement antagonist that is effective to substantially inhibit C3a receptor (C3aR) and/or C5a receptor (C5aR) signal transduction in dendritic cells (DCs) induced by TLR signaling as well as inhibit T cell and B cell immune responses.

In an aspect of the application, the at least one complement antagonist is selected from the group consisting of a small molecule, a polypeptide, and a polynucleotide. In some aspects, the polypeptide includes an antibody directed against at least one of C3, C5, C3 convertase, C5 convertase, C3a, C5a, C3aR, or C5aR. In other aspects, the polypeptide can include decay accelerating factor (DAF) (CD55) that accelerates the decay of C5 convertase and C3 convertase. In some aspects, the polynucleotide includes a small interfering RNA directed against a polynucleotide encoding at least one of C3, C5, C3aR, or C5aR.

Another aspect of the application relates to a method of treating a T cell and/or B cell mediated disorder in a subject. The method includes administering at least one complement antagonist to a subject having or suspected of having the T cell and/or B cell mediated disorder an amount of complement antagonist effective to substantially inhibit C3a receptor (C3aR) and/or C5a receptor (C5aR) signal transduction in dendritic cells (DCs) induced by TLR signaling as well as inhibit T cell and B cell immune responses.

In some aspects, the TLR, T cell, and/or B cell mediated disorder is selected from the group consisting of achlorhydra autoimmune active chronic hepatitis, acute disseminated encephalomyelitis, acute hemorrhagic leukoencephalitis, Addison's disease, agammaglobulinemia, alopecia areata, Alzheimer's disease, amyotrophic lateral sclerosis, ankylosing spondylitis, anti-gbm/tbm nephritis, antiphospholipid syndrome, antisynthetase syndrome, aplastic anemia, arthritis, atopic allergy, atopic dermatitis, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune polyendocrine syndrome, autoimmune progesterone dermatitis, autoimmune thrombocytopenia purpura, autoimmune uveitis, balo disease/balo concentric sclerosis, Bechets syndrome, Berger's disease, Bickerstaff's encephalitis, Blau syndrome, bullous pemphigoid, Castleman's disease, Chagas disease, chronic fatigue immune dysfunction syndrome, chronic inflammatory demyelinating polyneuropathy, chronic lyme disease, chronic obstructive pulmonary disease, Churg-Strauss syndrome, cicatricial pemphigoid, coeliac disease, Cogan syndrome, cold agglutinin disease, cranial arteritis, crest syndrome, Crohns disease, Cushing's syndrome, Dego's disease, Dercum's disease, dermatitis herpetiformis, dermatomyositis, diabetes mellitus type 1, Dressler's syndrome, discoid lupus erythematosus, eczema, endometriosis, enthesitis-related arthritis, eosinophilic fasciitis, epidermolysis bullosa acquisita, essential mixed cryoglobulinemia, Evan's syndrome, fibrodysplasia ossificans progressive, fibromyalgia, fibromyositis, fibrosing aveolitis, gastritis, gastrointestinal pemphigoid, giant cell arteritis, glomerulonephritis, Goodpasture's syndrome, Graves' disease, Guillain-barre syndrome (GBS), Hashimoto's encephalitis, Hashimoto's thyroiditis, henoch-schonlein purpura, hidradenitis suppurativa, Hughes syndrome, inflammatory bowel disease (IBD), idiopathic inflammatory demyelinating diseases, idiopathic pulmonary fibrosis, idiopathic thrombocytopenic purpura, iga nephropathy, inflammatory demyelinating polyneuopathy, interstitial cystitis, irritable bowel syndrome (IBS), Kawasaki's disease, lichen planus, Lou Gehrig's disease, lupoid hepatitis, lupus erythematosus, meniere's disease, microscopic polyangiitis, mixed connective tissue disease, morphea, multiple myeloma, multiple sclerosis, myasthenia gravis, myositis, narcolepsy, neuromyelitis optica, neuromyotonia, occular cicatricial pemphigoid, opsoclonus myoclonus syndrome, ord thyroiditis, Parkinson's disease, pars planitis, pemphigus, pemphigus vulgaris, pernicious anaemia, polymyalgia rheumatic, polymyositis, primary biliary cirrhosis, primary sclerosing cholangitis, progressive inflammatory neuropathy, psoriasis, psoriatic arthritis, raynaud phenomenon, relapsing polychondritis, Reiter's syndrome, rheumatoid arthritis, rheumatoid fever, sarcoidosis, schizophrenia, Schmidt syndrome, Schnitzler syndrome, scleritis, scleroderma, Sjogren's syndrome, spondyloarthropathy, sticky blood syndrome, still's disease, stiff person syndrome, sydenham chorea, sweet syndrome, takayasu's arteritis, temporal arteritis, transverse myelitis, ulcerative colitis, undifferentiated connective tissue disease, undifferentiated spondyloarthropathy, vasculitis, vitiligo, Wegener's granulomatosis, Wilson's syndrome, Wiskott-Aldrich syndrome, hypersensitivity reactions of the skin, atherosclerosis, ischemia-reperfusion injury, myocardial infarction, and restenosis.

Yet another aspect of the application relates to a method of treating TLR, T cell, and/or B cell mediated disorders in a subject. The method includes administering to the subject a therapeutically effective amount of at least one complement antagonist and a pharmaceutically acceptable carrier. The at least one complement antagonist can substantially inhibit interaction of at least one of C3a or C5a with the C3a receptor (C3aR) and C5a receptor (C5aR) on interacting dendritic cells and T cells in the subject. The at least one complement antagonist advantageously does not substantially inhibit innate systemic complement activation in the subject.

### BRIEF DESCRIPTION OF THE DRAWINGS

FIGS. 1A-D illustrate graphs showing TLR stimulation induces complement gene upregulation via autocrine C3ar1/C5ar1 signaling in DCs. (A) Purified OT-II CD4⁺ cells (1×10⁶) mixed with splenic CD11c=DCs (2.5×10⁵) were tested in IFN-g ELISPOT assays upon stimulation with various concentrations of OVA₃₂₃₋₃₃₉ (left panel, arrow indicates lowest concentration of Ag to initiate a response) or various PAMPs with (black bars) or without (white bars) Ag (right three panels, arrows indicate subthreshold levels of each PAMP that did not induce IFN-g production). Bars represent mean and SD of triplicates. Each experiment was repeated at least once. *p<0.05 compared with baseline. (B) Relative C3, fB, and fD gene expression (RT-PCR) of cultures containing OT-II cells plus splenic APCs from WT or various complement component-deficient animals, as indicated, with or without 0.1 μg/ml OVA₃₂₃₋₃₃₉, with or without 0.1 μg/ml poly I:C or 0.1 μg/ml LPS or 0.1 μg/ml CpG for 1 h. Each bar shows mean and SD of triplicate values and is representative of at least two individual experiments. *p<0.05 compared with unstimulated baseline. Relative expression (qPCR) of C3 (left panels) and fD (right panels) in RNA obtained from OT-II cells mixed with DCs from WT or Ticam1^{-/-} mice (C) or from WT or MyD88^{-/-} mice (D), as indicated, with or without 0.1 mg/ml OVA₃₂₃₋₃₃₉, with or without 0.1 mg/ml poly I:C or 0.1 mg/ml LPS. Each bar shows mean+SD of triplicate values and is representative of three individual experiments. *p>0.05.

FIGS. 2A-I illustrate graphs and plots showing TLR-induced enhancement of in vitro monoclonal and polyclonal T cell responses requires C3/C5 and autocrine C3ar1/C5ar1 signaling in immune cells. (A) Frequencies of IFN-g-producing OT-II cells (ELISPOT) when cultured with DCs from WT, C3^{-/-}, fD^{-/-}, C5ar1^{-/-}, C3ar1^{-/-}, or C3ar1^{-/-}C5ar1^{-/-} mice, as indicated, plus OVA³²³⁻³³⁹ (0.1 μg/ml), with or without LPS (0.1 μg/ml), CpG (0.5 μg/ml), or poly I:C (0.5 μg/ml). (B) IL-2 gene expression (qPCR) from cultures of OT-II cells mixed with OVA₃₂₃₋₃₃₉ (0.1 μg/ml) plus WT or C3ar1^{-/-C}5ar1^{-/-} DCs for 1 h. (CE)WT or C3ar1^{-/-}C5ar1^{-/-} splenic H-2^{b} DCs were prestimulated, with or without CpG overnight, as indicated, and cocultured with CFSE-labeled allogeneic naive WT H-2^{d} CD4⁺ T cells for 4 d. Representative CFSE dilution plots (C), quantified CFSE dilution (D), and live T cell numbers at the end of the culture period (E). (F and G) WT or C3^{-/-}C5def splenic H-2^{d} DCs were stimulated, with or without CpG overnight, as indicated, and cocultured with CFSE-labeled allogeneic WT or C3^{-/-} total CD4⁺ cells for 4 d. Quantified CFSE dilution (F) and live T cell numbers on day 4 (G). **p, 0.05 compared with unstimulated controls, *p<0.05 compared with stimulated WT counterparts, two-way ANOVA or unpaired t test. (H) Concentrations of C5a detected in 48-h culture supernatants of TEa CD4⁺ T cells mixed with allogeneic (bxd) F1 APCs or syngeneic B6 APCs, with or without CpG (10 mg/ml), as indicated. Assays were performed in serum-free media. (I) DAF expression on bxd F1 CD11c⁺ DCs and TEa CD4⁺ T cells in the absence or presence of CpG (10 μg/ml), as indicated, 48 h after initiating cultures. Representative flow plots (left panel) and quantification (right panel). Each experiment was repeated at least two or three times with similar results. Error bars indicate mean±SEM. *p<0.05, unpaired t test. ND, not detected; ns, not significant, p>0.05.

FIGS. 3A-G illustrate graphs and plots showingTLR9-induced DC maturation in vivo requires autocrine immune cell C3ar1/C5ar1 expression. (A and B) In vivo CpG titration. (A) Quantification of normalized changes in DC expression (flow cytometry, mean fluorescence intensity [MFI]) of MHCII, CD80, and CD86 on splenic DCs isolated 4 h after injection with various doses of CpG or vehicle control from H-2^{b} WT mice. (B) Quantification of 96-h CFSE dilution (left panel) and live cell numbers (right panel) of CFSE-labeled WT H-2^{d} T cells mixed with WT H-2^{b} DCs isolated 4 h after injection with various doses of CpG or control, gated on CD4⁺ T cells. Pooled data from two independently done experiments with similar results (each with two or three animals per group). Error bars indicate mean±SEM. *p<0.05 compared with unstimulated controls, unpaired t test. Representative flow plots and quantification of normalized changes in DC expression (flow cytometry, MFI) of CD80 (C) and CD86 (D) on splenic DCs isolated 4 h after injection with 100 mg of CpG or vehicle control from H-2^{b} WT versus C3ar1^{-/-}C5ar1^{-/-} mice. Representative 96-h CFSE-dilution plots (E) and quantified live cell numbers at the end of the cultures (F) of CFSE-labeled WT H-2d T cells mixed with WT or C3ar1^{-/-}C5ar1^{-/-} H-2^{b} DCs isolated 4 h after injection with CpG or control, gated on CD4+ or CD8+ T cells. (G) Relative gene expression (qPCR) of TNF-α, IL-1β, IL-12p40, and IL-6 in WT or C3ar1^{-/-}C5ar1^{-/-} splenic DCs isolated 4 h after injection with CpG or vehicle control. Each experiment was repeated at least three times with similar results (each time with two or three animals per group). Bars indicate mean±SEM. **p<0.05 compared with unstimulated controls, unpaired t test, *p<0.05, **p<0.01 compared with stimulated WT counterparts, two-way ANOVA. ns, not significant, p>0.05.

FIGS. 4A-F illustrate graphs and plots showing BM cellderived autocrine C3ar1/C5ar1 signaling mediates TLR9-induced DC maturation in vivo. (A) Representative flow plots (left panels) and quantification (right panel) of expression (flow cytometry, mean fluorescence intensity [MFI]) of C5ar1 on CD11c+ cells from recipient peripheral blood isolated 10 wk after WT→Myd88^{-/-} or C3ar1^{-/-}C5ar1^{-/-}→Myd88^{-/-} BM transplants. Representative flow plots and quantification of normalized changes in DC expression (flow cytometry, MFI) of CD80 (B) and CD86 (C) on splenic DCs isolated 4 h after injection with 100 mg of CpG or vehicle control from WT→Myd88^{-/-} or C3ar1^{-/-}C5ar1^{-/-}→Myd88^{-/-} BM chimeras. Quantified percentage of proliferation (D) and live cell numbers (E) on day 4 for CFSE-labeled WT H-2d T cells mixed with splenic DCs isolated from H-2b WT→Myd88^{-/-} or C3ar1^{-/-}C5ar1^{-/-}→Myd88^{-/-} BM chimeras 4 h after injection with CpG or control, gated on CD4⁺ or CD8⁺ T cells. (F) Relative gene expression (qPCR) of IL-1β, TNF-α, II-12p40, and IL-6 in the splenic DCs isolated 4 h after injection with CpG or vehicle control from the BM chimeras mentioned in (A)(E). Representative data from two independently performed experiments (each with two or three animals per group). Bars indicate mean±SEM. **p<0.05 compared with unstimulated controls, *p<0.05 compared with stimulated WT counterparts, unpaired t test or two-way ANOVA. ns, not significant, p>0.05.

FIGS. 5A-E illustrate graphs showing TLR9-induced DC maturation in vivo requires immune cell-derived complement proteins C3/C5. (A) C3 zymosan assay. Recipient sera from each BM chimera group were tested for the presence of serum C3 10 wk after BM transplants. Quantification of C3 deposition on zymosan particles (flow cytometry, mean fluorescence intensity [MFI]) is shown. (B) Normalized changes in DC expression (flow cytometry, MFI) of CD80 (left panel) and CD86 (right panel) on splenic DCs isolated 4 h after injection with 100 mg of CpG or vehicle control from H-2^{d} WT→C3^{-/-}C5def, C3^{-/-}C5^{def}→WT, WT→WT, and C3^{-/-}C5def→C3^{-/-}C5^{def} BM chimeras. Quantified percentage of proliferation (left panels) and live cell number (right panels) on day 4 for CFSE labeled WT H-2^{b} CD4⁺ (C) and CD8⁺ (D) T cells mixed with DCs isolated from H-2^{d} WT→C3^{-/-}C5def or C3^{-/-}C5def→WT BM chimeras 4 h after injection with CpG or vehicle control. E) Relative gene expression (qPCR) of IL-1β, TNF-α, IL-12p40, and IL-6 in splenic DCs isolated from H-2d WT→C3^{-/-}C5def or C3^{-/-}C5def→WT BM chimeras 4 h after injection with CpG or vehicle control (n=2-4 per group). Bars indicate mean±SEM. *p<0.05, two-way ANOVA or unpaired t test. ns, not significant, p>0.05.

FIGS. 6A-F illustrate autocrine C3ar1/C5ar1 signaling in DCs that regulates TLR9-induced changes in gene-expression pathways related to inflammation and immune cell signaling. RNA isolated from splenic DCs of WT or C3ar1^{-/-}C5ar1^{-/-} mice 4 h after injection with CpG (100 mg i.v.) or vehicle control was profiled by Affymetrix Mouse microarrays. (A) Venn diagram of CpG-induced upregulated genes found in WT DCs and/or C3ar1^{-/-}C5ar1^{-/-} DCs. (B) Heat map depicting the relative quantities of the top 60 of the 608 genes uniquely upregulated by CpG-stimulated WT DCs (n=3) compared with the WT DCs from untreated mice (n=2). (C) Heat map depicting relative quantities of the top 60 of the 590 genes upregulated by CpG in WT and C3ar1^{-/-}C5ar1^{-/-} DCs compared with their respective untreated controls (n=3 per group). (D) List of the top 10 most significant gene ontology terms (by p values) for the unique CpG-upregulated genes shown in (B) Database for Annotation, Visualization and Integrated Discovery (DAVID) analysis. List of the top 10 most significant gene ontology terms (E) and Kyoto Encyclopedia of Genes and Genomes pathways [(F), DAVID analysis] for genes shown in (C).

FIGS. 7A-E illustrate plots and graphs showing CpG-induced cardiac allograft rejection and enhanced alloimmunity in vivo are immune cell C3ar1/C5ar1 signaling dependent. (A) Survival of H-2d WT allografts transplanted into H-2b WT or C3ar1^{-/-}C5ar1^{-/-} recipients+MR1 (day 0), with or without CpG (days 1, 3, and 5), as indicated (n=6 per group). *p<0.01, log-rank (Mantel-Cox) test. Representative flow plots depicting the percentage of donor- reactive IFN-g-producing CD8⁺ T cells in the recipient spleen (B) with quantification (C, upper panel) and total number (C, lower panel) on day 14 posttransplant for WT or C3ar1^{-/-}C5ar1^{-/-} recipients of WT allografts treated with MR1, with or without CpG, as indicated. Pooled data from four independently done experiments with similar results (each with two or three animals per group) are shown. *p<0.05, unpaired t test. (D) Survival of WT H-2d hearts transplanted into H-2b C3ar1^{-/-}C5ar1^{-/-}→WT or WT→WT BM chimeras+MR1 (day 0) and CpG treatment (days 1, 3, and 5) (n=5 per group). (E) Survival of H-2b WT allografts transplanted into H-2^{d} WT or C3^{-/-C}5^{def} recipients, with or without MR1 (days 0 and 7), with or without CpG (days 1, 3, and 5), as indicated (n=9 per group for WT or C3^{-/-}C5^{def}→WT+MR1+CpG, n=3-5 per group for the rest). *p<0.01, log-rank (MantelCox) test. ns, not significant.

FIGS. 8A-E illustrate plots and graphs showing CpG-induced T_{reg} instability is C3ar1/C5ar1 dependent. (A) Schematic diagram of experimental design for (B and C) (left panel) and flow cytometry depiction of induced T_{regs} (Foxp3GFP⁺dTomato⁺) and ex-T_{regs} (Foxp3GFP^{neg}dTomato⁺) (right panel). Representative flow plots (B) and quantified conversion rates [(C), percentage ex-T_{reg}/total labeled T_{reg} population] gated on live CD4⁺ T cells in WT or C3ar1^{-/-}C5ar1^{-/-}T_{reg} fate-mapping mice on day 14 after allocardiac transplantation and subsequent MR1, with or without CpG treatment. Pooled data from five independently done experiments with similar results (each time with two to four animals per group) are shown. Bars indicate mean 6 SEM. (D) Schematic diagram of experimental design for (E). (E) Quantification of conversion rates (percentage of ex-T_{regs}/total labeled T_{reg} population) gated on splenic live CD4⁺ T cells in WT or C3ar1^{-/-}C5ar1^{-/-} hosts on day 7 after T_{reg} transfer and with or without CpG treatment. Pooled data from two independently done experiments with similar results (each time with two or three animals per group) are shown. Bars indicate mean±SEM. *p<0.05, unpaired t test. ns, not significant, p>0.05.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the present application.

In the context of the present application, the term “polypeptide” refers to an oligopeptide, peptide, or protein sequence, or to a fragment, portion, or subunit of any of these, and to naturally occurring or synthetic molecules. The term “polypeptide” also includes amino acids joined to each other by peptide bonds or modified peptide bonds, i.e., peptide isosteres, and may contain any type of modified amino acids. The term “polypeptide” also includes peptides and polypeptide fragments, motifs and the like, glycosylated polypeptides, and all “mimetic” and “peptidomimetic” polypeptide forms.

As used herein, the term “polynucleotide” refers to oligonucleotides, nucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, siRNAs, microRNAs, and ribonucleoproteins. The term also encompasses nucleic acids, i.e., oligonucleotides, containing known analogues of natural nucleotides, as well as nucleic acid-like structures with synthetic backbones.

As used herein, the term “antibody” refers to whole antibodies, e.g., of any isotype (IgG, IgA, IgM, IgE, etc.), and includes fragments thereof which are also specifically reactive with a target polypeptide. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain polypeptide. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term “antibody” also includes polyclonal, monoclonal, or other purified preparations of antibodies, recombinant antibodies, monovalent antibodies, and multivalent antibodies. Antibodies may be humanized, and may further include engineered complexes that comprise antibody-derived binding sites, such as diabodies and triabodies.

As used herein, the term “complementary” refers to the capacity for precise pairing between two nucleobases of a polynucleotide and its corresponding target molecule. For example, if a nucleobase at a particular position of a polynucleotide is capable of hydrogen bonding with a nucleobase at a particular position of a target polynucleotide (the target nucleic acid being a DNA or RNA molecule, for example), then the position of hydrogen bonding between the polynucleotide and the target polynucleotide is considered to be complementary. A polynucleotide and a target polynucleotide are complementary to each other when a sufficient number of complementary positions in each molecule are occupied by nucleobases, which can hydrogen bond with each other. Thus, “specifically hybridizable” and “complementary” are terms which can be used to indicate a sufficient degree of precise pairing or complementarity over a sufficient number of nucleobases such that stable and specific binding occurs between a polynucleotide and a target polynucleotide.

As used herein, the terms “effective,” “effective amount,” and “therapeutically effective amount” refer to that amount of a complement antagonist and/or a pharmaceutical composition thereof that results in amelioration of symptoms or a prolongation of survival in a subject with a TLR, T cell, and/or B cell mediated disorder. A therapeutically relevant effect relieves to some extent one or more symptoms of a TLR, T cell, and/or B cell mediated disorder, or returns to normal either partially or completely one or more physiological or biochemical parameters associated with or causative of a TLR, T cell, and/or B cell mediated disorder.

As used herein, the term “subject” refers to any warm-blooded organism including, but not limited to, human beings, rats, mice, dogs, goats, sheep, horses, monkeys, apes, rabbits, cattle, etc.

As used herein, the terms “complement polypeptide” or “complement component” refer to a polypeptide (or a polynucleotide encoding the polypeptide) of the complement system that functions in the host defense against infections and in the inflammatory process. Complement polypeptides constitute target substrates for the complement antagonists provided herein.

As used herein, the term “complement antagonist” refers to a polypeptide, polynucleotide, or small molecule capable of substantially reducing expression of C3, C5, C3a, C5a, C5aR, and/or C3aR in T cells or dendritic cells (DCs), substantially inhibiting C3aR and/or C5aR signal transduction of T cells, and/or substantially reducing interaction of C3a and C5a with C3aR and C5aR expressed by interacting dendritic cells (DCs) and T cells.

This application relates generally to a method of modulating Toll-like receptor (TLR) signaling in dendritic cells (DCs), and also to therapeutic methods of treating TLR, T cell, and/or B cell mediated disorders in a subject. It was found that autocrine C3ar1/C5ar1 signaling in DCs is a requisite process in TLR-mediated DC maturation required for the induction and amplification of T_{eff} cell responses. We found that TLR3, 4 and 9 ligations upregulates alternative pathway complement gene (i.e., C3 and fB) expression in splenic DCs in concert with downregulating cell surface DAF and that the process is MyD88- and/or TICAM1-dependent. The TLRs rapidly trigger activation of innate immune pathways in DCs and ultimately result in DC maturation that promotes T_{eff} cell activation and the production of pro-inflammatory IFNγ.

The example described herein shows that this TLR-initiated complement production and C3ar1/C5ar1 signaling by immune cells is required for TLR-induced a) increases in DC costimulatory molecules, b) pro-inflammatory cytokine secretion by DCs, c) upregulation of DC gene pathways broadly related to inflammation and immune responses, d) DC-dependent augmentation of T_{eff} proliferation/expansion, and e) T_{reg} instability. The example also shows that the absence of C3ar1/C5ar1 entirely prevented or severely blunted these changes that have been shown to be essential for induction of effective immune responses to pathogens, model antigens and autoantigens. Thus, immune cell-derived C3a/C3ar1 and C5a/C5ar1 interactions are crucial downstream intermediary steps between TLR-stimulation and DC maturation required for the development and amplification of T cell immune responses and support the use of complement inhibitors for treating TLR driven disorders or diseases.

Accordingly, in some embodiments, a method of treating a TLR, T cell, and/or B cell mediated disorder in a subject having or at risk of the TLR, T cell, and/or B cell mediated disorder includes administering to the subject an amount of at least one complement antagonist that is effective to substantially inhibit C3a receptor (C3aR) and/or C5a receptor (C5aR) signal transduction in dendritic cells (DCs) induced by TLR signaling as well as inhibit T cell and B cell immune responses.

In some embodiments, the complement antagonist can substantially inhibit the interaction of at least one of C3a or C5a with the C3a receptor (C3aR) and C5a receptor (C5aR) on the DCs to substantially inhibit C3a receptor (C3aR) and/or C5a receptor (C5aR) signal transduction in the DCs. In other embodiments, an inhibition or reduction in the functioning of a C3/C5 convertase can prevent cleavage of C5 and C3 into C5a and C3a, respectively. An inhibition or reduction in the functioning of C5a and C3a polypeptides can reduce or eliminate the ability of C5a and C3a to interact with C5aR and C3aR of DCs and substantially inhibit C3a receptor (C3aR) and/or C5a receptor (C5aR) signal transduction in the DCs. An inhibition or reduction in the functioning of a C5aR or C3aR may similarly reduce or eliminate the ability of C5a and C3a to interact C5aR and C3aR, respectively, and substantially inhibit C3a receptor (C3aR) and/or C5a receptor (C5aR) signal transduction in the DCs.

In some embodiments, the complement antagonist can include at least one of a C5a antagonist, a C3a antagonist, a C5aR antagonist, or a C3aR antagonist. It is also contemplated that more than one complement antagonist can be administered concurrently to DCs in order to inhibit C3a/C5a production and/or DC-T cell C3aR/C5aR signal transduction.

In some embodiments, the at least one complement antagonist can include various C5aR antagonists known in the art. For example, C5aR antagonists include those described by Short et al. (1999) Effects of a new C5a receptor antagonist on C5a- and endotoxin-induced neutropenia in the rat. British Journal of Pharmacology, 125:551-554, Woodruff et al. (2003) A Potent C5a Receptor Antagonist Protects against Disease Pathology in a Rat Model of Inflammatory Bowel Disease. The Journal of Immunology, 171:5514-5520, Sumichika et al. (2002) Identification of a Potent and Orally Active Non-peptide C5a Receptor Antagonist. The Journal of Biological Chemistry, 277(51):49403-49407, all of which are incorporated herein by reference.

In one embodiment, C5aR antagonist can include the peptidomimetic C5aR antagonist JPE-1375 (Jerini AG, Germany). C5aR antagonists can further include small molecules, such as CCX168 (ChemoCentryx, Mountain View, Calif.).

In other embodiments, the at least one complement antagonist can include an antibody or antibody fragment directed against a complement component that can affect or inhibit the formation of C3a and/or C5a (e.g., DAF, anti-C5 convertase, and anti-C3 convertase) and/or reduce C5a/C3a-C5aR/C3aR interactions (e.g., anti-C5a, anti-C3a, anti-C5aR, C3aR antibodies).

In still other embodiments, the at least one complement antagonist can include an antibody or antibody fragment directed against a complement component that can affect or inhibit the formation of C3a and/or C5a (e.g., anti-Factor B, anti-Factor D, anti-C5, anti-C3, anti-C5 convertase, and anti-C3 convertase) and/or reduce C5a/C3a-C5aR/C3aR interactions (e.g., anti-C5a, anti-C3a, anti-C5aR, and C3aR antibodies). In one example, the antibody or antibody fragment can be directed against or specifically bind to an epitope, an antigenic epitope, or an immunogenic epitope of a C5, C3, C3a, C5a, C5aR, C3aR, C5 convertase, and/or C3 convertase. The term “epitope” as used herein can refer to portions of C5, C3, C3a, C5a, C5aR, C3aR, C5 convertase, and/or C3 convertase having antigenic or immunogenic activity. An “immunogenic epitope” as used herein can include a portion of a C5, C3, C3a, C5a, C5aR, C3aR, C5 convertase, and/or C3 convertase that elicits an immune response in a subject, as determined by any method known in the art. The term “antigenic epitope” as used herein can include a portion of a polypeptide to which an antibody can immunospecifically bind as determined by any method well known in the art.

Examples of antibodies directed against C5, C3, C3a, C5a, C5aR, C3aR, C5 convertase, and/or C3 convertase are known in the art. For example, mouse monoclonal antibodies directed against C3aR can include those available from Santa Cruz Biotechnology, Inc. (Santa Cruz, Calif.). Monoclonal anti-human C5aR antibodies can include those available from Research Diagnostics, Inc. (Flanders, N.J.). Monoclonal anti-human/anti-mouse C3a antibodies can include those available from Fitzgerald Industries International, Inc. (Concord, Me.). Monoclonal anti-human/anti-mouse C5a antibodies can include those available from R&D Systems, Inc. (Minneapolis, Minn.).

In another aspect of the application, the at least one complement antagonist can include purified polypeptide that is a dominant negative or competitive inhibitor of C5, C3, C3a, C5a, C5aR, C3aR, C5 convertase, and/or C3 convertase. As used herein, “dominant negative” or “competitive inhibitor” refers to variant forms of a protein that inhibit the activity of the endogenous, wild type form of the protein (i.e., C5, C3, C3a, C5a, C5aR, C3aR, C5 convertase, and/or C3 convertase). As a result, the dominant negative or competitive inhibitor of a protein promotes the “off” state of protein activity. In the context of the present application, a dominant negative or competitive inhibitor of C5, C3, C3a, C5a, C5aR, C3aR, C5 convertase, and/or C3 convertase is a C5, C3, C3a, C5a, C5aR, C3aR, C5 convertase, and/or C3 convertase polypeptide, which has been modified (e.g., by mutation of one or more amino acid residues, by posttranscriptional modification, by posttranslational modification) such that the C5, C3, C3a, C5a, C5aR, C3aR, C5 convertase, and/or C3 convertase inhibits the activity of the endogenous C5, C3, C3a, C5a, C5aR, C3aR, C5 convertase, and/or C3 convertase.

In some embodiments, the competitive inhibitor of C5, C3, C3a, C5a, C5aR, C3aR, C5 convertase, and/or C3 convertase can be a purified polypeptide that has an amino acid sequence, which is substantially similar (i.e., at least about 75%, about 80%, about 85%, about 90%, about 95% similar) to the wild type C5, C3, C3a, C5a, C5aR, C3aR, C5 convertase, and/or C3 convertase but with a loss of function. The purified polypeptide, which is a competitive inhibitor of C5, C3, C3a, C5a, C5aR, C3aR, C5 convertase, and/or C3 convertase, can be administered to a DCs.

It will be appreciated that antibodies directed to other complement components used in the formation of C5, C3, C5a, C3a, C5 convertase, and/or C3 convertase can be used in accordance with the method of the present application to reduce and/or inhibit interactions C5a and/or C3a with C5aR and C3aR between dendritic cells and T cells. The antibodies can include, for example, known Factor B, properdin, and Factor D antibodies that reduce, block, or inhibit the classical and/or alternative pathway of the complement system.

In a further aspect of the present application, the at least one complement antagonist can include RNA interference (RNAi) polynucleotides to induce knockdown of an mRNA encoding a complement component. For example, an RNAi polynucleotide can comprise a siRNA capable of inducing knockdown of an mRNA encoding a C3, C5, C5aR, or C3aR polypeptide in the DC.

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response.

As used herein, the term “RNAi construct” is a generic term used throughout the specification to include small interfering RNAs (siRNAs), hairpin RNAs, and other RNA species, which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (I) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences.

The choice of promoter and other regulatory elements generally varies according to the intended host cell. In general, expression vectors of utility in recombinant DNA techniques are often in the form of “plasmids” which refer to circular double stranded DNA loops, which, in their vector form are not bound to the chromosome. In the present specification, “plasmid” and “vector” are used interchangeably as the plasmid is the most commonly used form of vector. However, the application is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the application has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA Likewise, bases may be modified to block the activity of adenosine deaminase. The RNAi construct may be produced enzymatically or by partial/total organic synthesis, any modified ribonucleotide can be introduced by in vitro enzymatic or organic synthesis.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Examples of a siRNA molecule directed to an mRNA encoding a C3a, C5a, C5aR, or C3aR polypeptide are known in the art. For instance, human C3a, C3aR, and C5a siRNA is available from Santa Cruz Biotechnology, Inc. (Santa Cruz, Calif.). Additionally, C5aR siRNA is available from Qiagen, Inc. (Valencia, Calif.). siRNAs directed to other complement components, including C3 and C5, are known in the art.

In other embodiments, the RNAi construct can be in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects, which may be caused by the sequence-independent dsRNA response. In such embodiments, the use of local delivery systems and/or agents, which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid can be used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present application provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

In some embodiments, a lentiviral vector can be used for the long-term expression of a siRNA, such as a short-hairpin RNA (shRNA), to knockdown expression of C5, C3, C5aR, and/or C3aR in CD4⁺ T cells and dendritic cells. Although there have been some safety concerns about the use of lentiviral vectors for gene therapy, self-inactivating lentiviral vectors are considered good candidates for gene therapy as they readily transfect mammalian cells.

It will be appreciated that RNAi constructs directed to other complement components used in the formation of C5, C3, C5a, C3a, C5 convertase, and/or C3 convertase can be used in accordance with the method of the present application to reduce and/or inhibit interactions between C5a and/or C3a with C5aR and C3aR on the FoxP3⁺ Treg cells. The RNAi constructs can include, for example, known Factor B, properdin, and Factor D siRNA that reduce expression of Factor B, properdin, and Factor D.

Moreover, it will be appreciated that other antibodies, small molecules, and/or peptides that reduce or inhibit the formation of C5, C3, C5a, C3a, C5 convertase, and/or C3 convertase and/or that reduce or inhibit interactions C5a and/or C3a with C5aR and C3aR on naive CD4⁺ cells can be used as a complement antagonist in accordance with the method of the present application. These other complement antagonists can be administered to the subject and/or naive CD4⁺ T cells at amount effective to generate CD4⁺FoxP3⁺ Treg cells. Example of such other complement antagonists include C5aR antagonists, such as AcPhe[Orn-Pro-D-cyclohexylalanine-Trp-Arg, prednisolone, and infliximab (Woodruff et al,. The Journal of Immunology, 2003, 171: 5514-5520), hexapeptide MeFKPdChaWr (March et al., Mol Pharmacol 65:868-879, 2004), PMX53 and PMX205, and N-[(4-dimethylaminophenyl)methyl]-N-(4-isopropylphenyl)-7-methoxy-1,2,3,4-tetrahydronaphthalen-1-carboxamide hydrochloride (W-54011) (Sumichika et al., J. Biol. Chem., Vol. 277, Issue 51, 49403-49407, Dec. 20, 2002), and a C3aR antagonist, such as SB 290157 (Ratajczak et al., Blood, 15 Mar. 2004, Vol. 103, No. 6, pp. 2071-2078).

The at least one complement antagonist can be administered to the DCs in vivo or ex vivo. In some embodiments, at least one complement antagonist can be administered to DCs or a subject at an amount that is effective to substantially inhibit C3a receptor (C3aR) and/or C5a receptor (C5aR) signal transduction in dendritic cells (DCs) induced by TLR signaling as well as inhibit T cell and B cell immune responses. Inhibition of T cell and B cell immune responses can be used to prevent local and systemic organ and tissue destruction in cell therapies aimed at alleviating T cell and/or B cell mediated disorders or diseases.

The term “T cell mediated disease”, “T cell mediated disorder”, “B cell mediated disease”, and/or “B cell mediated disorder” refers to diseases and disorders in which an aberrant immune reaction involves T cell-mediated immune mechanisms and/or B cell-mediated immune mechanisms, as opposed to humoral immune mechanisms. T cell mediated disorders and/or B cell mediated disorders contemplated by the present application also include T cell mediated and/or B cell autoimmune diseases or disorders. The language “autoimmune disorder” is intended to include disorders in which the immune system of a subject reacts to autoantigens, such that significant tissue or cell destruction occurs in the subject. The term “autoantigen” is intended to include any antigen of a subject that is recognized by the immune system of the subject. The terms “autoantigen” and “self-antigen” are used interchangeably herein. The term “self” as used herein is intended to mean any component of a subject and includes molecules, cells, and organs. Autoantigens may be peptides, nucleic acids, or other biological substances.

Thus, the methods of the application pertain to treatments of immune disorders in which tissue destruction is primarily mediated through activated T cells, B cells, and immune cells. For example, the methods of the present application can be used in the treatment of autoimmune conditions or diseases, such as inflammatory diseases, including but not limited to achlorhydra autoimmune active chronic hepatitis, acute disseminated encephalomyelitis, acute hemorrhagic leukoencephalitis, Addison's disease, agammaglobulinemia, alopecia areata, Alzheimer's disease, amyotrophic lateral sclerosis, ankylosing spondylitis, anti-gbm/tbm nephritis, antiphospholipid syndrome, antisynthetase syndrome, aplastic anemia, arthritis, atopic allergy, atopic dermatitis, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune polyendocrine syndrome, autoimmune progesterone dermatitis, autoimmune thrombocytopenia purpura, autoimmune uveitis, balo disease/balo concentric sclerosis, Bechets syndrome, Berger's disease, Bickerstaff's encephalitis, blau syndrome, bullous pemphigoid, Castleman's disease, Chagas disease, chronic fatigue immune dysfunction syndrome, chronic inflammatory demyelinating polyneuropathy, chronic lyme disease, chronic obstructive pulmonary disease, Churg-Strauss syndrome, cicatricial pemphigoid, coeliac disease, Cogan syndrome, cold agglutinin disease, cranial arteritis, crest syndrome, Crohns disease, Cushing's syndrome, Dego's disease, Dercum's disease, dermatitis herpetiformis, dermatomyositis, diabetes mellitus type 1, Dressler's syndrome, discoid lupus erythematosus, eczema, endometriosis, enthesitis-related arthritis, eosinophilic fasciitis, epidermolysis bullosa acquisita, essential mixed cryoglobulinemia, Evan's syndrome, fibrodysplasia ossificans progressive, fibromyalgia, fibromyositis, fibrosing aveolitis, gastritis, gastrointestinal pemphigoid, giant cell arteritis, glomerulonephritis, Goodpasture's syndrome, Graves' disease, Guillain-barre syndrome (gbs), Hashimoto's encephalitis, Hashimoto's thyroiditis, henoch-schonlein purpura, hidradenitis suppurativa, Hughes syndrome, inflammatory bowel disease (IBD), idiopathic inflammatory demyelinating diseases, idiopathic pulmonary fibrosis, idiopathic thrombocytopenic purpura, iga nephropathy, inflammatory demyelinating polyneuopathy, interstitial cystitis, irritable bowel syndrome (ibs), Kawasaki's disease, lichen planus, Lou Gehrig's disease, lupoid hepatitis, lupus erythematosus, meniere's disease, microscopic polyangiitis, mixed connective tissue disease, morphea, multiple myeloma, multiple sclerosis, myasthenia gravis, myositis, narcolepsy, neuromyelitis optica, neuromyotonia, occular cicatricial pemphigoid, opsoclonus myoclonus syndrome, ord thyroiditis, Parkinson's disease, pars planitis, pemphigus, pemphigus vulgaris, pernicious anaemia, polymyalgia rheumatic, polymyositis, primary biliary cirrhosis, primary sclerosing cholangitis, progressive inflammatory neuropathy, psoriasis, psoriatic arthritis, raynaud phenomenon, relapsing polychondritis, Reiter's syndrome, rheumatoid arthritis, rheumatoid fever, sarcoidosis, schizophrenia, Schmidt syndrome, Schnitzler syndrome, scleritis, scleroderma, Sjogren's syndrome, spondyloarthropathy, sticky blood syndrome, still's disease, stiff person syndrome, sydenham chorea, sweet syndrome, takayasu's arteritis, temporal arteritis, transverse myelitis, ulcerative colitis, undifferentiated connective tissue disease, undifferentiated spondyloarthropathy, vasculitis, vitiligo, Wegener's granulomatosis, Wilson's syndrome, Wiskott-Aldrich syndrome as well as hypersensitivity reactions of the skin, atherosclerosis, ischemia-reperfusion injury, myocardial infarction, and restenosis. The methods of the present application can also be used for the prevention or treatment of the acute rejection of transplanted organs where administration of a therapeutic described herein, may occur during the acute period following transplantation or as long-term post transplantation therapy.

In some embodiments, the DC can include a DC in the subject and the complement antagonist can be used to treat a T cell mediated disease and/or B cell mediated disease in the subject. The at least one complement antagonist can be administered to the subject to treat the T cell mediated disease and/or B cell mediated in the subject using any one or combination of known techniques.

In one aspect of the application, the complement antagonist can be administered directly or locally to a site of T cell mediated disease and/or B cell mediated disease in the subject. Local or direct administration of the complement antagonist into and/or about the periphery of the disease site is advantageous because the complement antagonist localizes at the disease site being treated and does not substantially affect the subject's innate complement system.

In another aspect of the application, the complement antagonist can be administered to the subject systemically by, for example, intravenous, intraarterial, intraperitoneal, intramuscular, subcutaneous, intrapleural, intrathecal, oral or nasal route, to treat the T cell mediated disease and/or B cell mediated disease or related disorder in the subject. When administered systemically, the complement antagonist can be targeted to a disease site to ensure that the complement antagonist does not adversely affect other normal cells expressing C5aR and/or C3aR, and to potentially mitigate adverse systemic effects on the subject's complement system. Several systems have been developed in order to restrict the delivery of the complement antagonist to the disease site. With the identification of cells specific receptors and antigens on mammalian cells, it is possible to actively target the complement antagonist using ligand or antibody bearing delivery systems. Alternatively, the complement antagonist can be loaded on a high capacity drug carriers, such as liposomes or conjugated to polymer carriers that are either directly conjugated to targeting proteins/peptides or derivatised with adapters conjugated to a targeting moiety.

Examples of antibodies which can be potentially conjugated to the complement antagonist to target the complement antagonist to the T cell mediated disease and/or B cell mediated disease site include, but are not limited to, anti-CD20 antibodies (e.g., Rituxan, Bexxar, Zevalin), anti-Her2/neu antibodies (e.g., Herceptin), anti-CD33 antibodies (e.g., Mylotarg), anti-CD52 antibodies (e.g., Campath), anti-CD22 antibodies, anti-CD25 antibodies, anti-CTLA-4 antibodies, anti-EGF-R antibodies (e.g. Erbitux), anti-VEGF antibodies (e.g. Avastin, VEGF Trap) anti-HLA-DR10β antibodies, anti-MUC1 antibodies, anti-CD40 antibodies (e.g. CP-870,893), anti-Treg cell antibodies (e.g., MDX-010, CP-675,206), anti-GITR antibodies, anti-CCL22 antibodies, and the like.

The complement antagonist, whether administered locally and/or systemically, can also be provided in a pharmaceutically acceptable composition. The phrase “pharmaceutically acceptable” should be understood to mean a material which is not biologically or otherwise undesirable, i.e., the material may be incorporated into an antiviral composition and administered to a subject without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. Sterile phosphate-buffered saline is one example of a pharmaceutically acceptable carrier. Other suitable carriers are well-known to those in the art. See, for example, REMINGTON'S PHARMACEUTICAL SCIENCES, 19th Ed. (1995), and later editions.

In general, the dosage of the at least one complement antagonist will vary depending upon such factors as the subject's age, weight, height, sex, general medical condition and previous medical history. Typically, it is desirable to provide the subject with a dosage of the at least one complement antagonist which is in the range of from about 1 pg/kg to 10 mg/kg (amount of agent/body weight of patient), although a lower or higher dosage also may be administered as circumstances dictate. The specific dosage or amount of complement antagonist administered to a DCswill be that amount effective to reduce or inhibit C5a/C3a-C5aR/C3aR interactions.

In an example of the method, a therapeutically effective amount of a pharmaceutical composition comprising a first antibody directed against C3aR and a second antibody directed against C5aR can be administered to DCs or a subject having a TLR, T cell, and/or B cell mediated disorder. The pharmaceutical composition can be administered to the subject intravenously using, for example, a hypodermic needle and syringe. Upon administration of the pharmaceutical composition to the subject, the first and second antibodies can respectively bind to C3aR and C5aR on at least one DC. Binding of the first and second antibodies can effectively inhibit or reduce the ability of C3a and C5a to respectively bind C3aR and C5aR. Consequently, C5a/C3a-C5aR/C3aR signaling can be reduced or eliminated.

The following examples are for the purpose of illustration only and are not intended to limit the scope of the claims, which are appended hereto.

### EXAMPLE

We found that, early during interaction of murine DCs with cognate T cells, both partners locally generate C3a and C5a, which establish autocrine C3ar1/C5ar1 signaling loops with DC- and T cell-expressed receptors (C3ar1 and C5ar1). This process occurs in concert with downregulation of the cell surface C3/C5 convertase regulator decay accelerating factor (DAF; or CD55). The resultant C3ar1/C5ar1 signaling in the DCs and cognate T cell partners plays an integral role in DC-induced Teff activation, including during GVHD.

The observations that C3ar1/C5ar1 signaling and TLR signals each cause changes in DCs that promote their ability to induce T_{eff} responses prompted the hypothesis that the two processes are linked (i.e., that TLR-enhancing effects on T_{eff} immunity are dependent on C3ar1/C5ar1 signal transduction in DCs). We used analyses of DC-interacting T cells, along with murine models of alloimmunity, to demonstrate that C3ar1/C5ar1 signaling in DCs is a requisite process connecting TLR stimulation with DC maturation and T_{eff} activation at threshold PAMP concentrations that model physiological TLR signaling.

### Materials and Methods

### Mice

C57BL/6 (B6), B6 CD45.1, B6 C3^{-/-}, OTII, B6 Myd88^{-/-}, B6 TRIF^{-/-}, and BALB/c mice were originally purchased from The Jackson Laboratory and maintained at the Mount Sinai Center for Comparative Medicine or at Case Western Reserve University. Factor D^{-/-} (fD^{-/-}) mice were a kind gift from Y. Xu (University of Alabama at Birmingham, Birmingham, Ala.). B10.D2 Hc⁰ mice (C5 deficient [C5def]; The Jackson Laboratory) and BALB/c C3^{-/-} mice (backcrossed for >12 generations from B6 C3^{-/-} mice) were crossed together to produce C3^{-/-}C5def mice. B6 C3ar1^{-/-}C5ar1^{-/-}, C3ar1^{-/-}C5ar1^{-/-}Foxp3-GFP mice were produced as previously described. Rosa(dTomato)XFoxp3CreERT2-GFP mice were obtained from A. Rudensky (Sloan-Kettering Institute, New York, N.Y.) and were crossed with B6 C3ar1^{-/-}C5arl^{-/-} mice to produce C3ar1^{-/-C}5ar1^{-/-} (dTomato)XFoxp3CreERT2-GFP mice. TEa mice (41) were a gift of J. Bromberg (University of Maryland). All mice were housed in the Icahn School of Medicine at Mount Sinai Center for Comparative Medicine or at Case Western Reserve University in accordance with guidelines of the Association for Assessment and Accreditation of Laboratory Animal Care International. All experiments were performed using animals that were littermates or were maintained in the same room and/or were cohoused within the same cages to limit the potential effects of microbiome differences.

### Abs and reagents

Abs against CD4, CD8, CD11c, and fixable viability dye (eBioscience), CD55 (BD Biosciences), CD88 (AbD Serotec), CD80, CD86, and MHCII (Miltenyi Biotec, Auburn, Calif.) were used for flow cytometry. CFSE was obtained from eBioscience. MR1 (anti-mouse CD40L) was purchased from Bio X Cell (West Lebanon, N.H.). Peptides were synthesized by GenScript (Piscataway, N.J.).

### Cell Isolation

Spleen was passed through a 40-mm strainer (BD Falcon) and lysed with RBC lysis buffer (Life Technologies/Thermo Scientific, Waltham Mass.). T cell depletion from bone marrow (BM) suspensions and isolation of murine splenic naive CD4⁺ T cells and total T cells/APCs were accomplished using magnetic beads and an autoMACS Pro Separator (Miltenyi Biotec). For splenic DC isolation, single-cell suspensions of spleen cells were treated with Collagenase D (Roche) for 30 min at 37° C. and incubated with CD11c MicroBeads (Miltenyi Biotec), following the manufacturer's protocol. The purity of DCs after isolation was 85-95%. Isolated DCs were stimulated with CpG ODN 1826 or LPS (both from InvivoGen) for 18 h in 96-well plates, if needed.

### Mixed Lymphocyte Responses

Splenic DCs from untreated mice were stimulated in vitro with TLR ligands overnight, washed with PBS three times, and then cocultured with naive allogeneic CD4⁺ T cells or unfractionated T cells labeled with CFSE for MLRs. Analogous studies were performed using splenic DCs isolated 4 h after i.v. injections of CpG. On day 4, CFSE dilution was assessed for cellular proliferation, and live cell numbers were counted in the well using flow cytometry. Cells were incubated in complete medium (RPMI 1640+10% FCS+L-glutamine+sodium pyruvate+nonessential amino acids+Pen/Strep+2-ME) at 37° C. Splenic DCs were phenotyped for surface markers by flow cytometry. In some experiments, cells were harvested at 18 h for analysis of cytokine gene expression by quantitative PCR (qPCR).

### ELISPOT Assays

Cytokine ELISPOT assays were performed using spleen cells cocultured with BALB/c APCs on IFN-g capture plates for 24 h and then analyzed as previously described.

### Flow Cytometry

Data were collected on a FACSCanto II (BD Biosciences) and analyzed using FlowJo software (TreeStar, Ashland, Oreg.) or Cytobank (Cytobank, Santa Clara, Calif.). To measure recall immune responses posttransplant, spleen cells from heart transplant recipients were stimulated with donor cells overnight and then analyzed for intracellular IFN-g within the CD4 or CD8 gate by flow cytometry.

### Heart Transplant

Heterotopic heart transplants were performed as previously described by our laboratory. For graft survival experiments, recipients were treated with anti-CD40L (anti-CD154) mAb MR1 (1 mg on day 0 and 500 mg on days 7 and 14, i.p.) with or without CpG ODN 1826 (100 mg on day 1 and 50 mg on days 3 and 5, i.p.). Heart graft function was monitored every other day by palpation; rejection was defined as the day on which a palpable heartbeat was no longer detectable and was confirmed by histology.

### Real-time PCR

RNA isolation was performed using TRIzol Reagent (Thermo Fisher), and cDNA was reverse transcribed using a High-Capacity cDNA Reverse Transcription Kit (Applied Biosystems), as per the manufacturer's instructions. Real-time PCR (TaqMan probes; Applied Biosystems) was performed using a CFX96 Real-Time PCR Detection System (Bio-Rad Laboratories). All mouse PCR primers were purchased from Life Technologies. PCR products were normalized to the control gene (GAPDH) and expressed as the fold increase compared with unstimulated cells using the DDCt method.

### C5a ELISA

Splenic APCs were cultured in serum-free HL-1 medium with allogeneic or syngeneic splenic T cells, with or without CpG (10 mg), in 48-well plates for 48 h. Culture supernatant fluids were concentrated with an Amicon Ultra-0.5, normal m.w. limit of 10 kDa (Millipore), and tested for C5a with a Mouse Complement Component C5a DuoSet ELISA (R&D Systems, Minneapolis, Minn.), as per the manufacturer's instructions.

### BM Chimeras

Six- to eight-week-old male B6 or BALB/c mice were fasted for 24 h prior to irradiation. On day 0, recipients were irradiated with 650 rad twice, with a $3-h interval between treatments. Once irradiated, mice received adoptive transfer of T cell-depleted BM cells isolated from the various donors. Eight to ten weeks later, the percentage of chimerism was assessed by flow cytometry.

### Tamoxifen Treatment and Treg Fate Mapping

Tamoxifen (Sigma-Aldrich) was dissolved in olive oil (Fluka) to a final concentration of 20 mg/ml by shaking overnight at 37° C. in a light-blocking vessel. The dose of tamoxifen was determined by weight: ˜75 mg/kg body weight of a mouse.

### Microarrays and Analysis

We isolated splenic CD11c⁺ DCs (using Miltenyi Biotec magnetic beads) from wild-type (WT) or C3ar1^{-/-}C5ar1^{-/-} mice 4 h after injection with CpG (100 mg) or vehicle control. The cells were immediately placed in TRIzol Reagent and sent to the State University of New York Albany Center for Functional Genomics. Total RNA was isolated by standard techniques (>150 pg RNA was obtained per sample). After quality-control testing, the samples were processed using standard WT Pico protocols and hybridized to Mouse Gene 2.0 ST Arrays, and the chips were scanned using a GeneChip Scanner 7G (all from Affymetrix). The intensity data at the probe set level were extracted and normalized with the RMA algorithm, and data quality was assessed using Expression Console (Affymetrix). The Affymetrix control probe sets or the probe sets with low intensity across all samples were excluded from downstream analysis. The limma test was performed on normalized data between comparison groups, and the differentially expressed genes with p, 0.05 were identified and visualized using a heat map. Gene ontology enrichment analysis using a Fisher exact test was performed on differentially expressed genes to investigate their associated biological functions or pathways. The microarray data presented in this article have been submitted to the National Center for Biotechnology Information Gene Expression Omnibus under accession number GSE98315 (https://www.ncbi.nlm.nih.gov/geo/query/acc.cgi? acc=GSE98315).

### Statistics

Statistical significance was determined using a Student t test (unpaired, two-tailed), two-way ANOVA (with a Bonferroni posttest to compare replicate means), or a log-rank (MantelCox) test with GraphPad Prism 5 or Prism 6, with a significance threshold p value, 0.05. All experiments were repeated at least twice. Data are presented as mean values with SD.

### Results

### TLR Signaling Requires Endogenous Immune Cell Complement Production

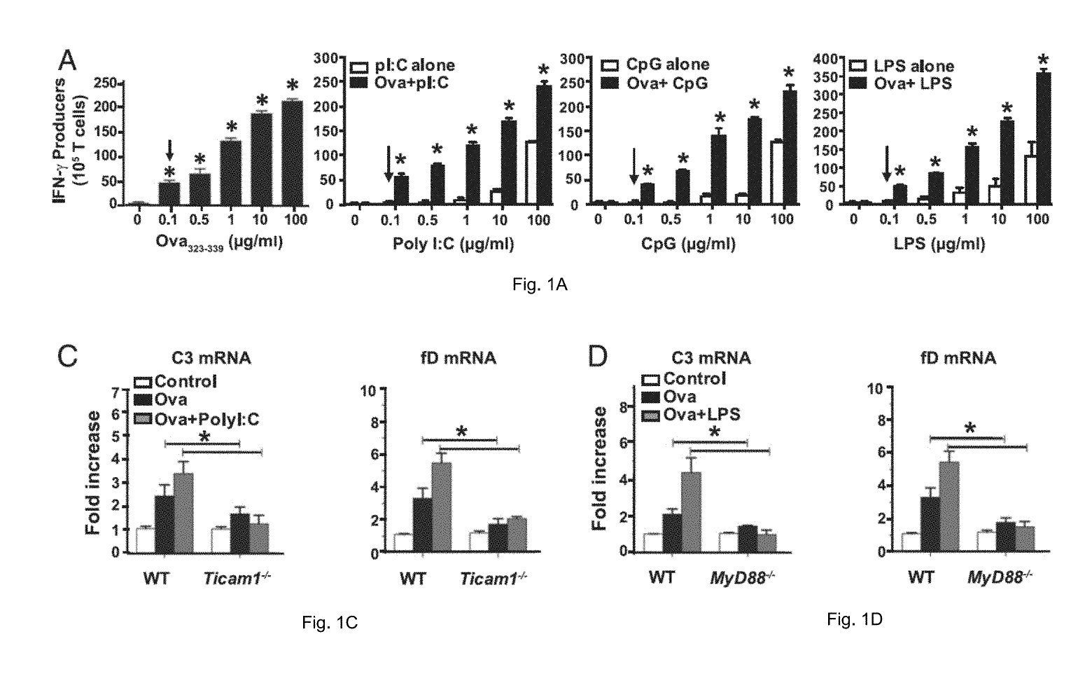

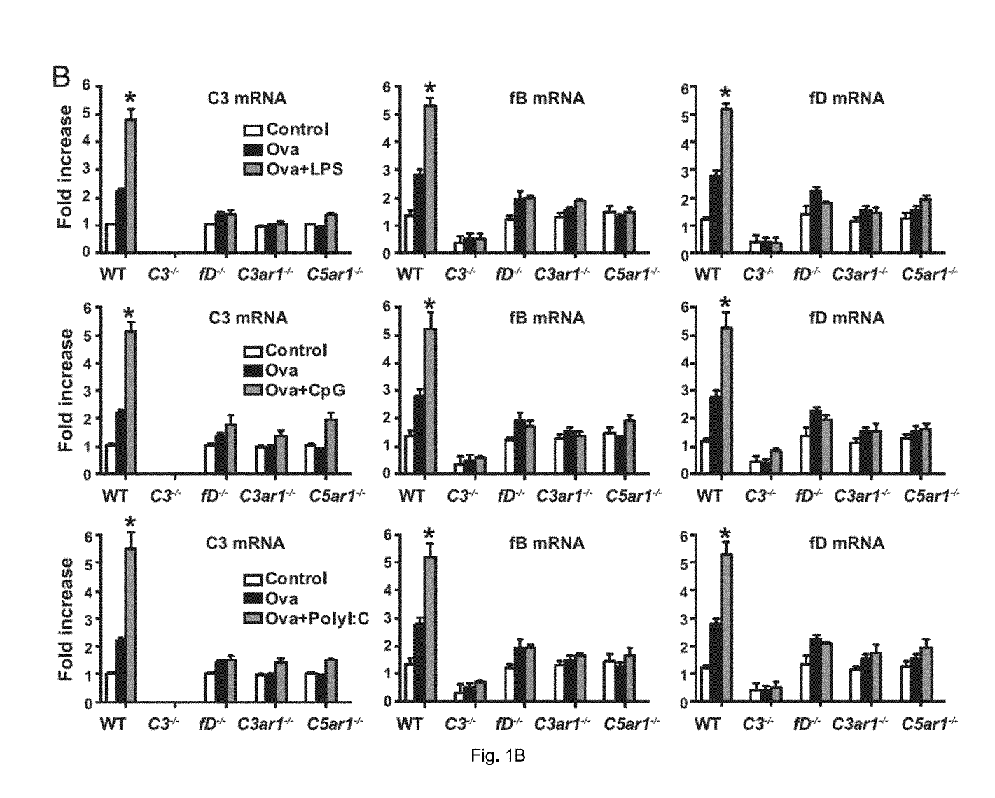

We first studied the effects of DC-produced complement on TLR3, TLR4, and TLR9 induction of IFN-γ production by OVA₃₂₃₋₃₃₉+I-A^{b} specific CD4⁺ OT-II T cells. To exclude the effects of systemic complement on signaling through each of the TLRs, we performed in vitro studies using DCs as APCs in the absence of serum complement. We titrated OVA₃₂₃₋₃₃₉ and TLR ligand concentrations to establish threshold conditions for detection of IFN-g by the responding cells (FIG. 1A). A total of 0.1 mg/ml OVA gave ˜25% of the maximal IFN-g response, and 0.1 mg/ml of polyinosinicpolycytidylic acid (poly I:C), CpG, or LPS did not generate IFN-g-producing cells in the absence of OVA₃₂₃₋₃₃₉ (FIG. 1A). In the presence of Ag, the number of IFN-γ-producing OT-II cells increased in a dose-dependent manner with the addition of increasing amounts of each PAMP (FIG. 1A), such that the effect of TLR signaling on endogenous complement production, and vice versa, could be interrogated.

Using the above conditions, with or without threshold amounts of each PAMP, we determined the effects of TLR stimulation on complement component gene expression by cells within the culture (noting that DCs produce ˜1000-fold more complement than T cells). The inclusion of each PAMP increased C3, factor B (fB), and factor D (fD) mRNA expression levels from OT-II cultures (FIG. 1B) over the levels induced by OVA323-339 without a PAMP. Substitution of C3^{-/-}, fD^{-/-}C3ar1^{-/-}, or C5ar1^{-/-} DCs for WT DCs abolished the TLR-induced upregulation (FIG. 1B), connecting autocrine C3ar1/C5ar1 signaling with TLR signaling. Studies with DCs from MyD88^{-/-} or Ticam1^{-/-} (Trif^{-/-}) mice showed that poly I:C-induced C3/fD (FIG. 1C) mRNA upregulation is mediated via Ticam1, that LPS-induced DC C3/fD (FIG. 1D) gene upregulation is mediated via MyD88, and that CpG-induced C3 expression required MyD88 (data not shown), together confirming that the PAMP/TLR-induced effects are transmitted through established canonical signaling pathways for each of the TLRs.

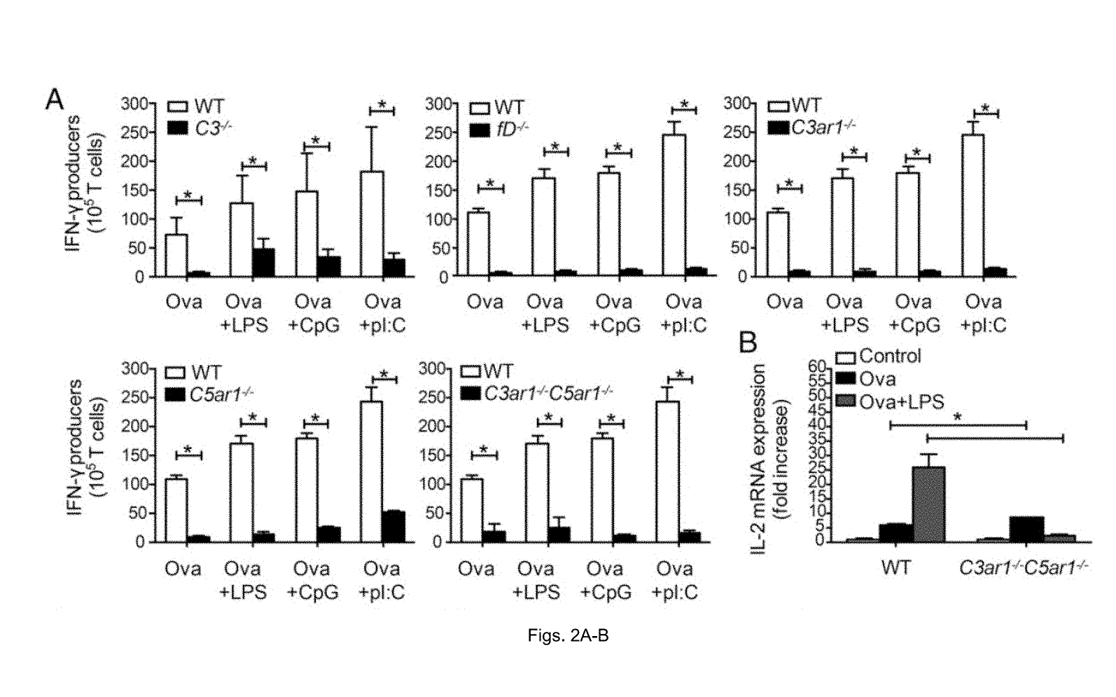

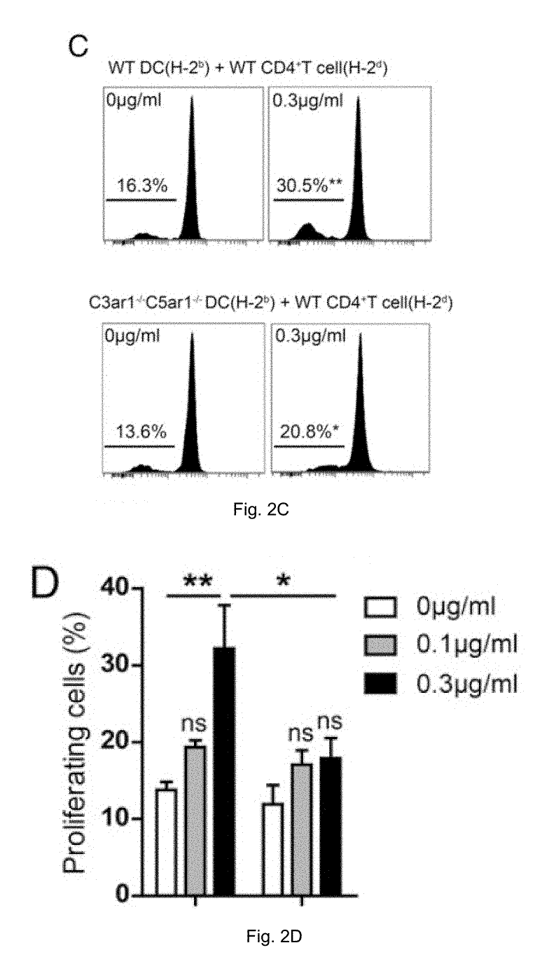

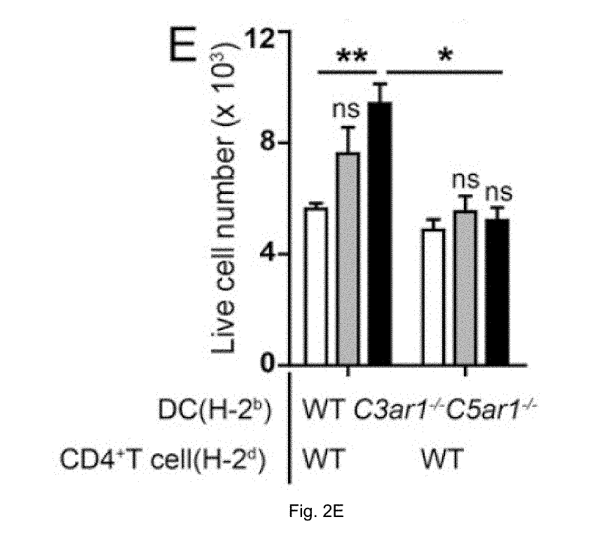

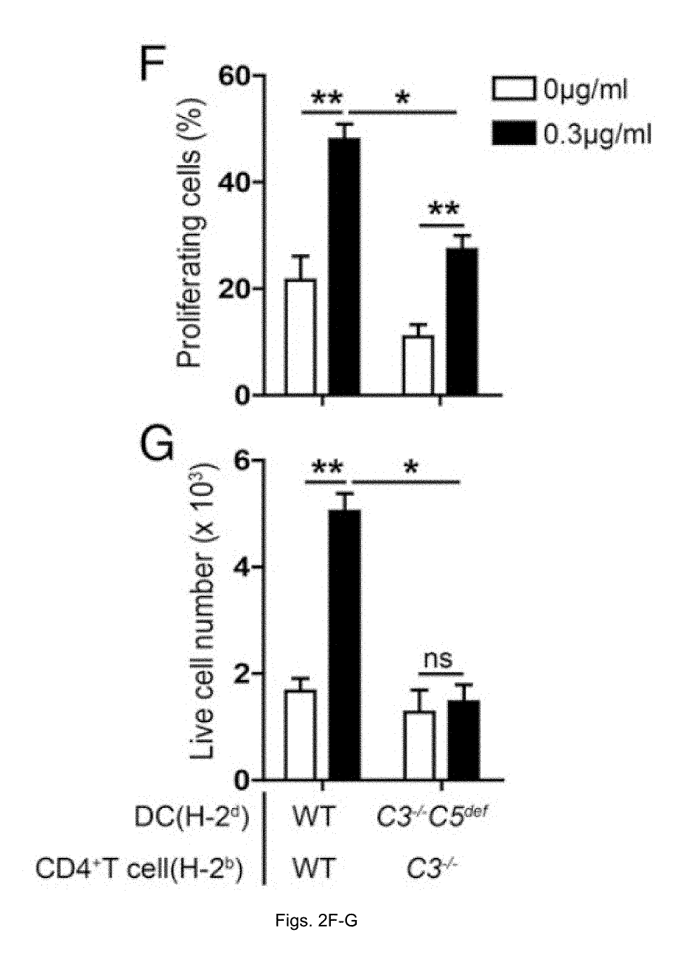

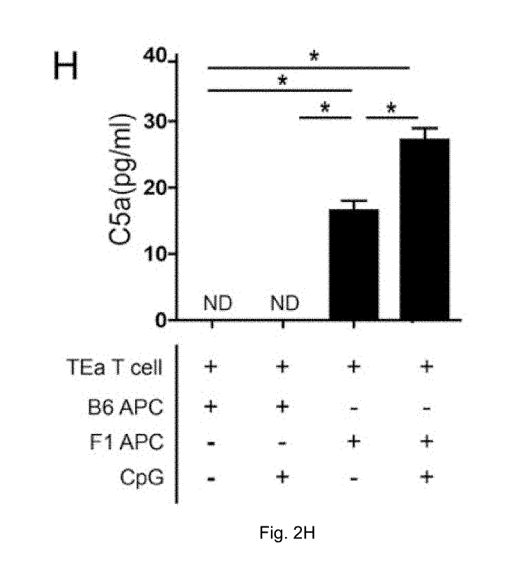

To test whether the TLR-induced increases in T cell IFN-g production and autocrine C3ar1/C5ar1 signaling in DCs are linked, we cultured OT-II cells with the defined threshold concentrations of Ag and PAMPs, using WT DCs or DCs from C3^{-/-}, fD^{-/-}, C3ar1^{-/-}, C5ar1^{-/-}, or C3ar1^{-/-}C5ar1^{-/-} mice (FIG. 2A). These assays showed that the absence of DCs reduced IFN-γ production by OT-II cells by 0.80%. The same pattern was observed for IL-2 RNA production by LPS-stimulated OT-II cells (FIG. 2B).

To verify that the link between TLR-induced T cell responses and DC-derived complement has relevance in a polyclonal system, we performed studies with B6 DCs and allogeneic BALB/c T cells, using T cell proliferation (CFSE dilution) and expansion (live cell number) as readouts (FIG. 2C-E). We determined that pretreatment with 0.3 mg/ml CpG was the minimum threshold dose required for WT DCs to increase alloreactive T cell proliferation and expansion (data not shown) in MLRs. Unstimulated allogeneic DCs induced proliferation of allogeneic T cells: 13-16% of the T cells in the culture underwent more than four cell divisions on day 4, consistent with previous observations. Although CpG pretreatment of WT DCs augmented alloreactive T cell proliferation and expansion (FIG. 2C-E), CpG pretreatment of C3ar1^{-/-}C5ar1^{-/-} DCs had no effect. Effects paralleling those with C3ar1^{-/-}C5ar1^{-/-} DCs occurred using C3/C5-deficient DCs (FIG. 2F, 2G). In the case of WT DCs, CpG induced stronger allogeneic T cell proliferation/expansion of purified CD4⁺ T cells (note reversal of responder/stimulator strains). For BALB/c H-2d C3^{-/-}C5def DCs pretreated with CpG and cultured with purified C3^{-/-} T cells (removing all C3 and DC-derived C5), proliferation was severely blunted, and T cell expansion was fully abrogated (FIG. 2F, 2G). The same was true for purified CpGinduced enhancements of CD8⁺ T cell responses and for LPSinduced enhancements of T cell proliferation/expansion (data not shown). Thus, for monoclonal T cells and polyclonal T cells responding to DCs in two different genetic backgrounds, TLR augmentation of T cell immunity in vitro was dependent on DC complement synthesis and C3ar1/C5ar1 signals.

To establish that the above interpretation applies in a disease-relevant context, we used a monoclonal TCR-transgenic system germane to alloimmune responses [i.e., TEa CD4³⁰ cells (reactive to I-A^{b}+I-E^{d}α₅₂₋₆₈), which have been used in transplant models]. We incubated TEa T cells with allogeneic (bxd)F1 APCs (that express I-A^{b}+I-E^{d}α₅₂₋₆₈) or syngeneic APCs as controls, with or without added CpG. (FIG. 2H). C5a was detectable in 48-h supernatants of cultures containing allogeneic (bxd)F1 APCs (which undergo cognate interactions with TEa cells). The amounts of C5a increased in cultures containing added CpG. In contrast, no C5a was detected in cultures containing control B6 APCs, with or without CpG. Flow cytometric analyses showed that CpG caused ˜30-50% downregulation of DAF on (bxd)F1 APCs and TEa CD4⁺ compared with cells incubated with media alone (FIG. 2I). Together with previous findings using polyclonal T cells (26), these data show that TLR-induced immune cell C5a production is connected to repression of DAF expression.

### Immune Cell C3ar1/C5ar1 Signaling Mediates TLR9-Induced DC maturation In Vivo

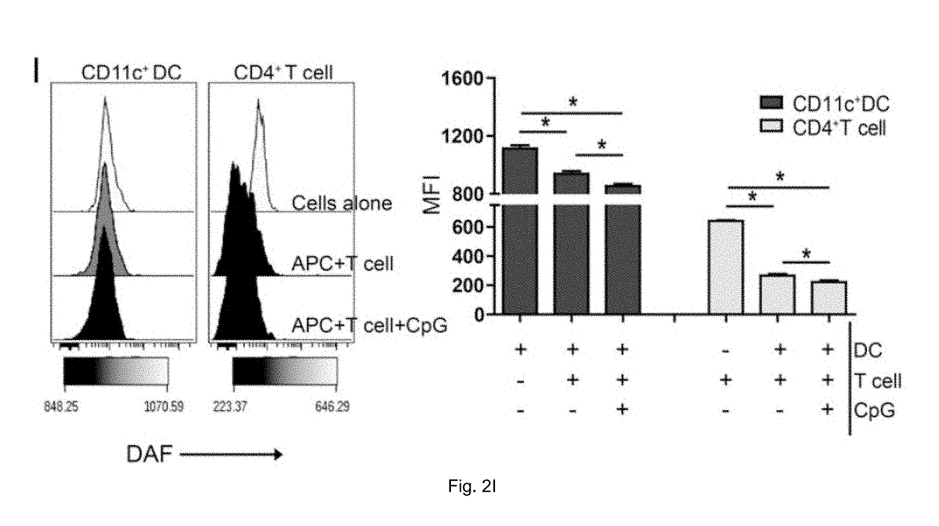

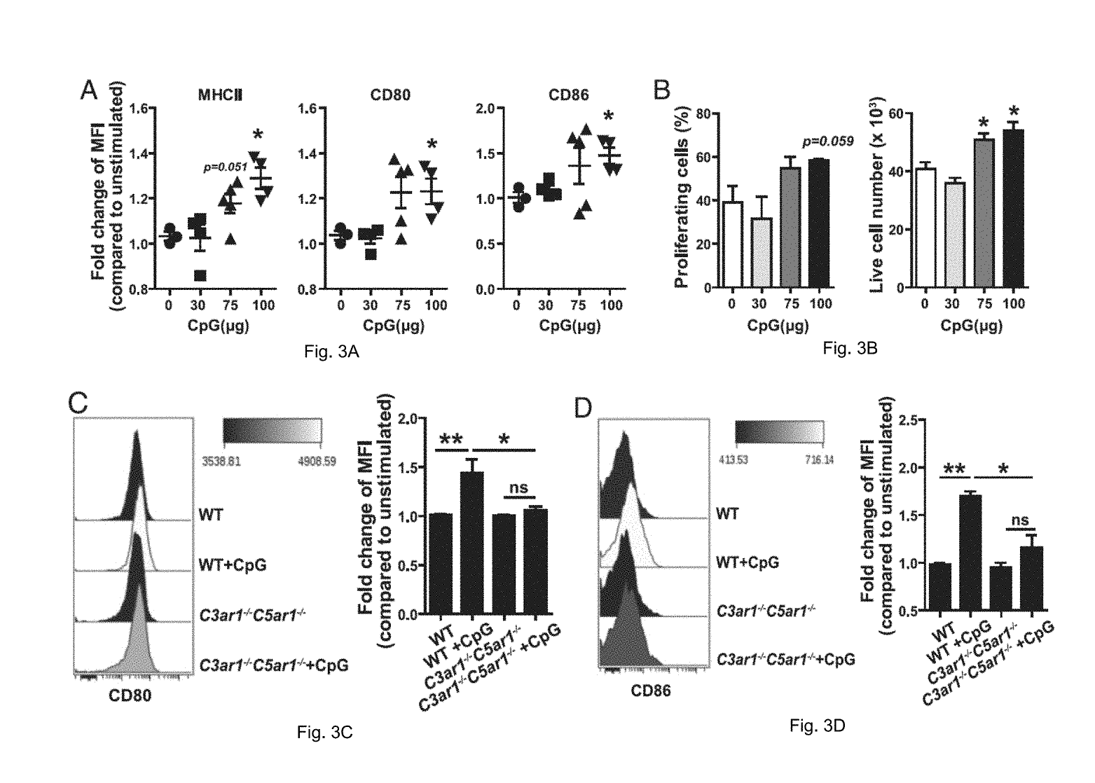

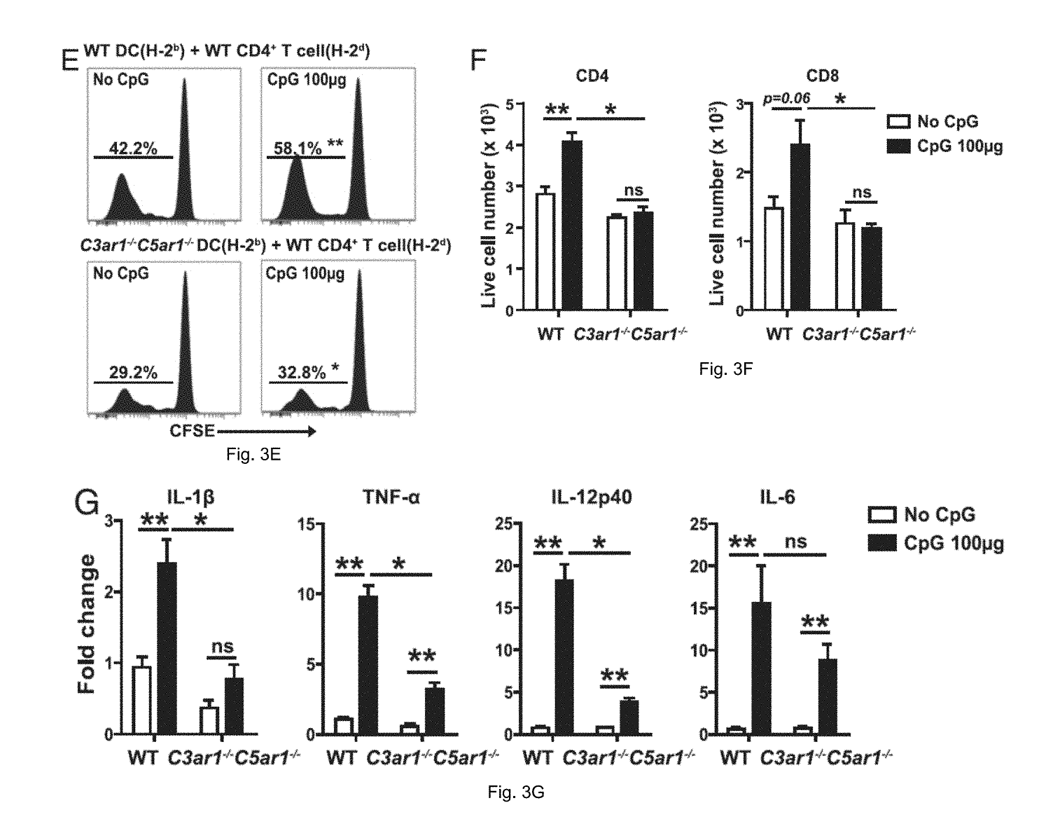

We next tested whether the interposition of C3ar1/C5ar1 signaling between TLR signals and DC maturation applies in vivo. We used TLR9 ligation, because its ligation by CpG has been extensively studied in GVHD and solid organ transplant systems. TLR9 stimulation also augments T_{eff} induction following immunization, as well as heightens pathology in several autoimmune models. To define the threshold concentrations of CpG required to induce DC maturation markers and allogeneic T cell stimulatory activity in vivo, we injected (i.v.) various amounts of CpG (0-100 μg) into WT mice. We isolated splenic CD11c⁺ DCs 4 h later and analyzed DC surface phenotypes and tested DC allostimulatory function by coculturing the DCs with allogeneic CFSE-labeled T cells (FIG. 3A, 3B). These assays demonstrated that 100 mg of CpG was the minimum concentration that consistently upregulated CD80, CD86, and MHCII on DC surfaces and enhanced proliferation and expansion of cocultured allogeneic T cells. We next injected groups of B6 WT and C3ar1^{-/-}C5ar1^{-/-} mice with 100 mg of CpG, isolated splenic CD11c⁺ DCs 4 h later, and compared DC surface phenotypes and function ex vivo between the two groups (FIG. 3C-G). These experiments showed that the absence of C3ar1/C5ar1 prevented CpG-induced upregulation of CD80/CD86 (FIG. 3C, 3D) and MHCII (WT: 40±2.8% increase versus no CpG; C3ar1^{-/-}C5ar1^{-/-}: 21±5.0%, p, 0.05 versus WT, data not shown), as well as abrogated CpG-induced augmentation of allogeneic T cell proliferation and expansion (FIG. 3E, 3F). CD40 expression levels were not altered by CpG administration in WT or C3ar1^{-/-}C5ar1^{-/-} DCs (data not shown). For WT DCs, CpG administration augmented mRNA expression of IL-1b, TNF-α, IL-12p40, and IL-6. In contrast, DCs from C3ar1/C5ar1 mice showed diminished TNF-α, IL-12p40, and IL-6 and fully restrained upregulation of IL-1β (FIG. 3G).

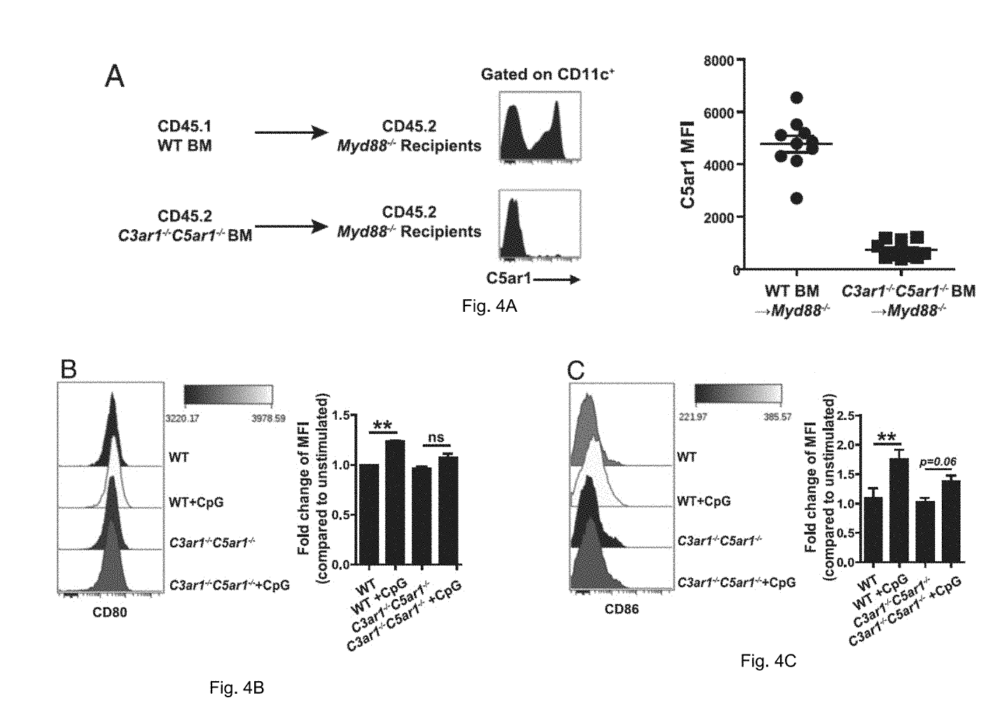

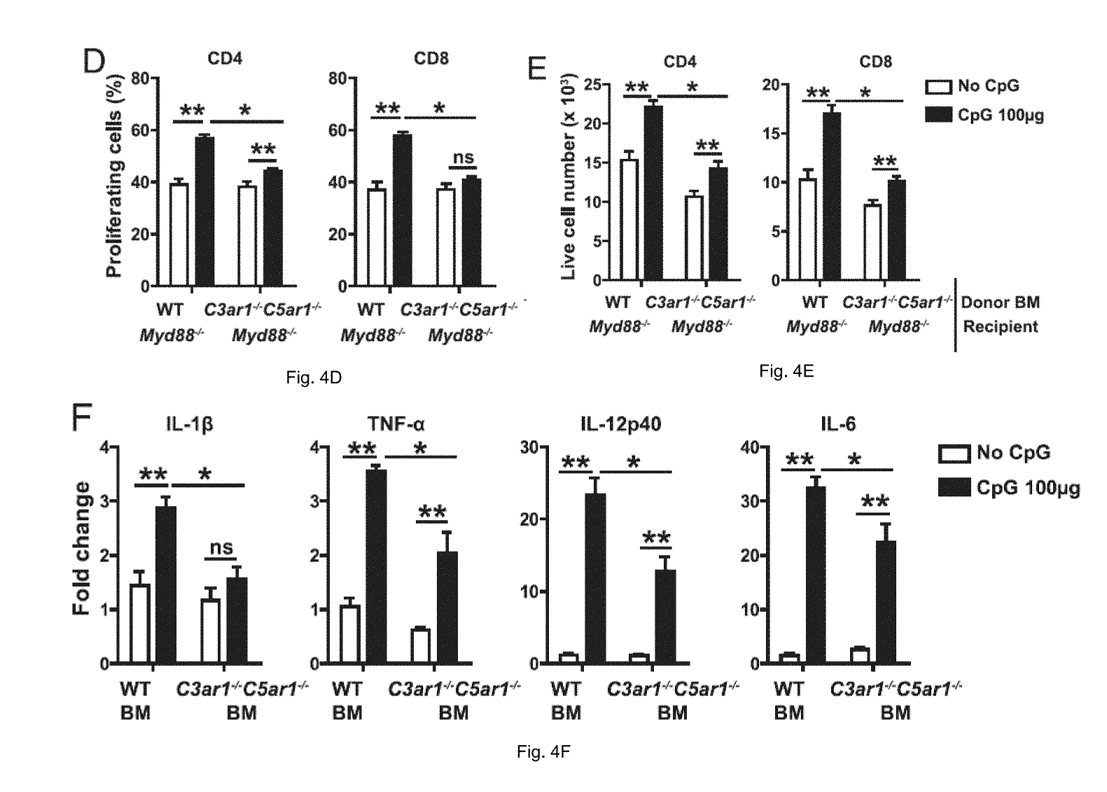

We next performed two sets of studies to exclude any contribution of C3ar1/C5ar1 or MyD88 signaling in non-BM cells. We transplanted BM from B6 WT or C3ar1^{-/-}C5ar1^{-/-} mice into congenic lethally irradiated B6 MyD88^{-/-} recipients. Because CpG/TLR9 signaling is MyD88 dependent, in these chimeras only the transplanted BM-derived cells are capable of responding to CpG through MyD88. Nine weeks later (after confirming chimerism, FIG. 4A), we injected groups of chimeras with 100 mg of CpG and analyzed DC phenotypes and function (FIG. 4B-F). These assays demonstrated that the added CpG induced maturation of WT DCs, as assessed by CD80/CD86 surface expression, the ability to stimulate alloreactive T cells, and the ability to upregulate proinflammatory cytokine gene expression, whereas it had much lesser effects on C3ar1^{-/-}C5ar1^{-/-} DCs.

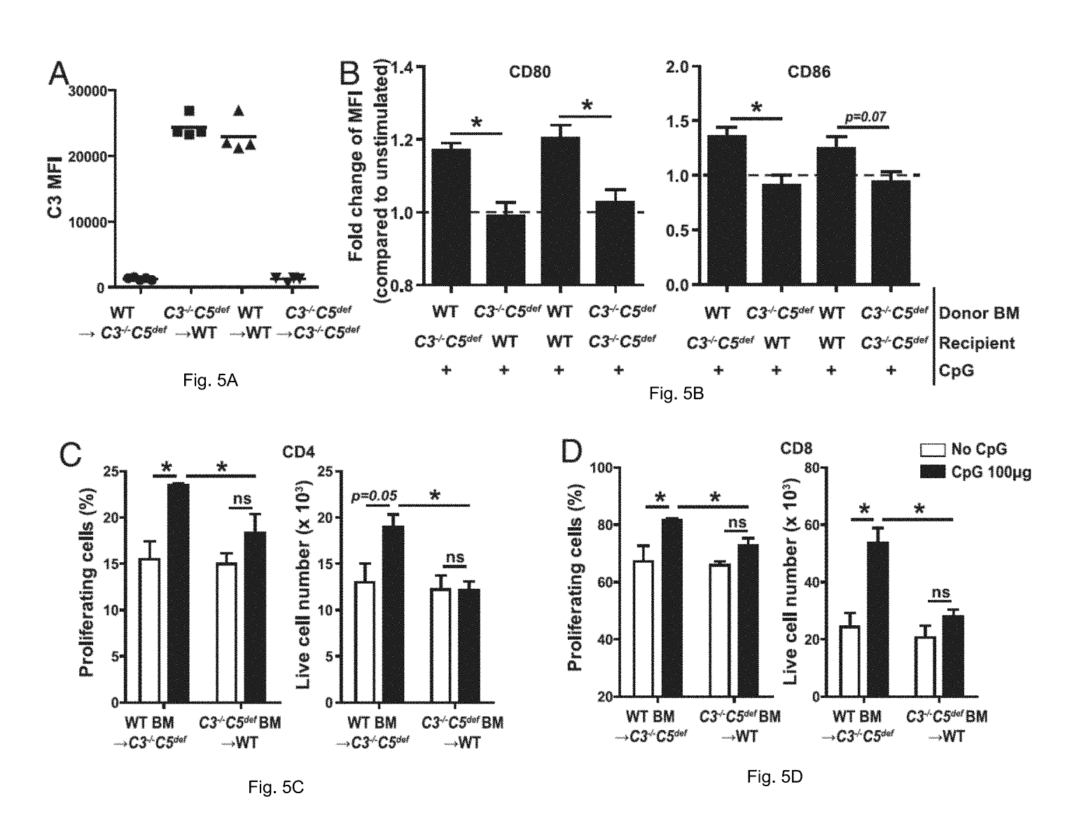

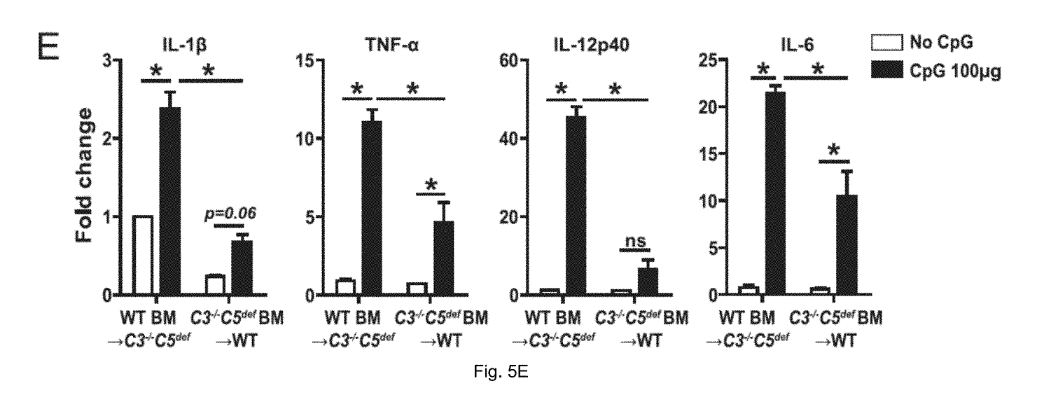

In the second approach, we tested whether the TLR effects conform to our prior findings that T_{eff} responses (without TLR stimulation) depend upon immune cell-derived complement rather than serum/liver-derived complement. We constructed WT BM→C3^{-/-}C5^{def}, C3^{-/-}C5^{def} BM→WT, and control WT→WT and C3^{-/-}C5^{def}→C3^{-/-}C5^{def} BM chimeras (H-2d) and verified 0.90% donor chimerism 10 wk later (FIG. 5A). We then injected groups of chimeras with CpG or vehicle, isolated splenic DCs 4 h later, and analyzed them as above (FIG. 5B-E). These assays showed that, although DCs from CpG-treated WT BM chimeras upregulated surface expression of CD80/86, these costimulatory molecules remained unchanged on DCs from CpG-treated chimeras possessing C3^{-/-}C5^{def} BM, regardless of host C3/C5 expression (FIG. 5B). Culturing of the isolated DCs with allogeneic T cells (FIG. 5C, 5D) showed that in vivo CpG administration augmented ex vivo T cell proliferation/expansion when DCs from mice with WT BM were used, but not when DCs from mice with C3^{-/-}C5def BM were used, regardless of serum complement. The absence of C3/C5 in BM cells likewise blunted CpG-induced upregulation of IL-1β, TNF-α, IL-12p40, and IL-6 RNA (FIG. 5E).

### CpG-Induced Changes in DC Transcriptomes are Dependent upon C3ar1/C5ar1 Signaling

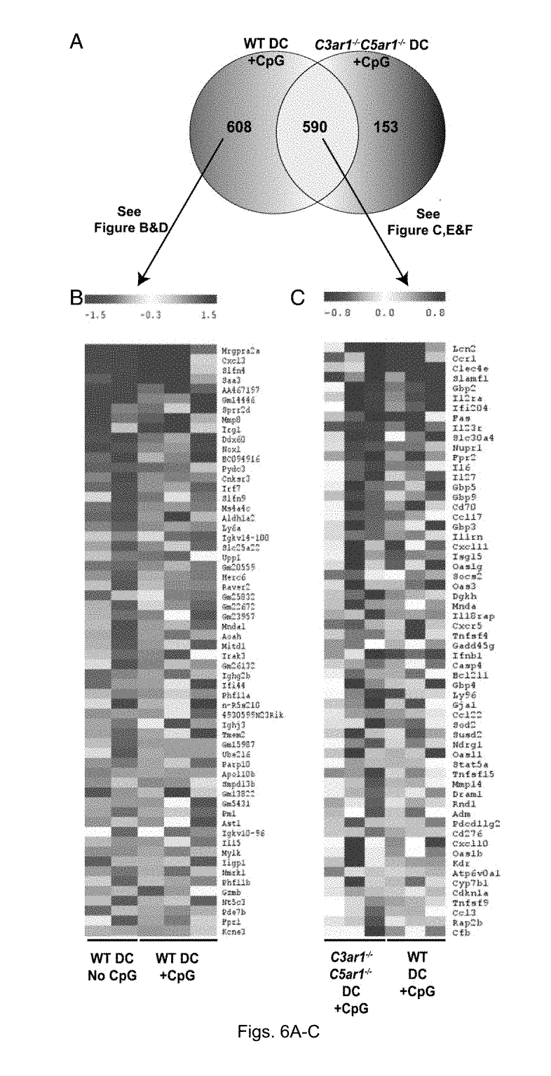

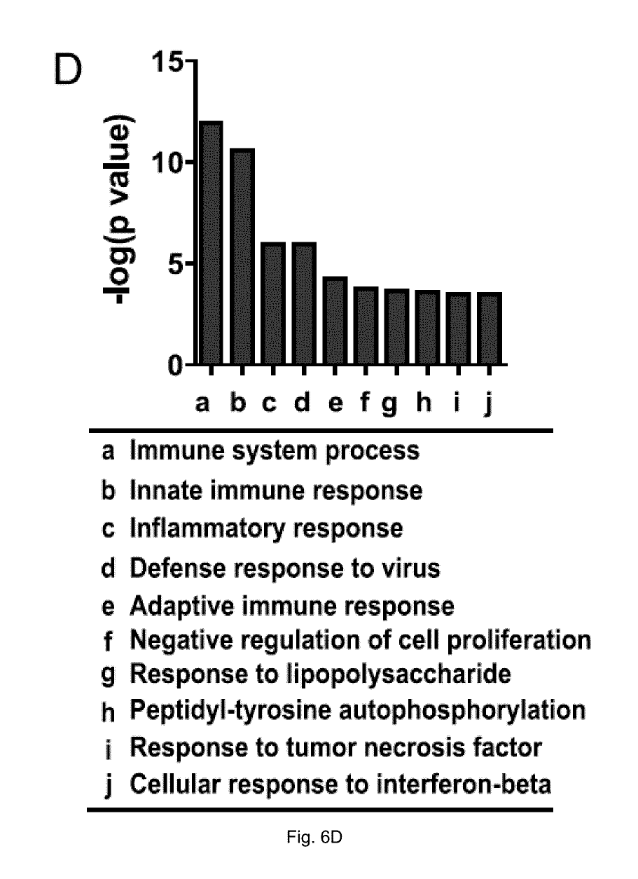

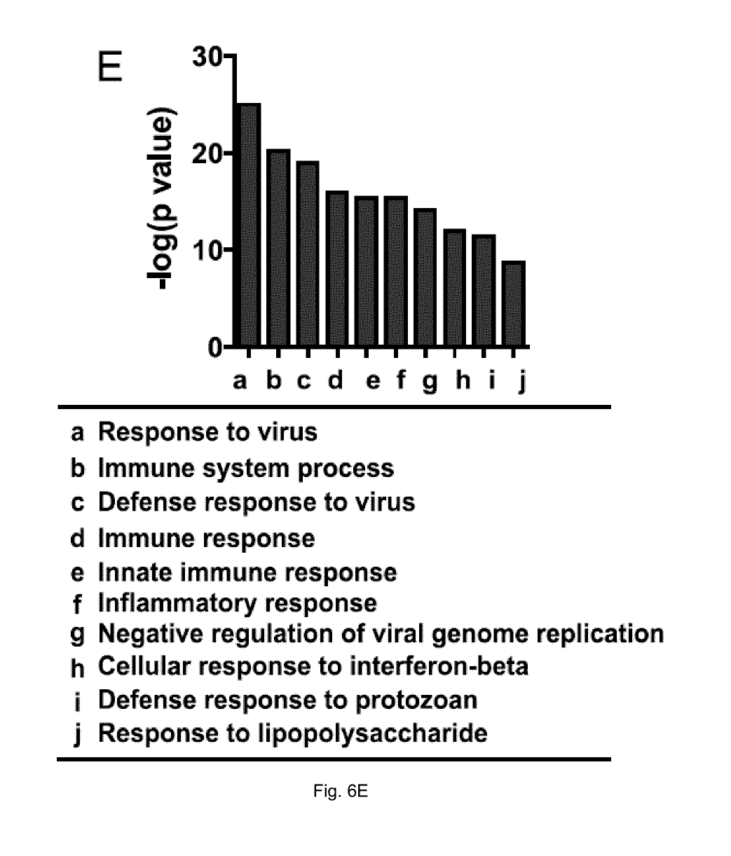

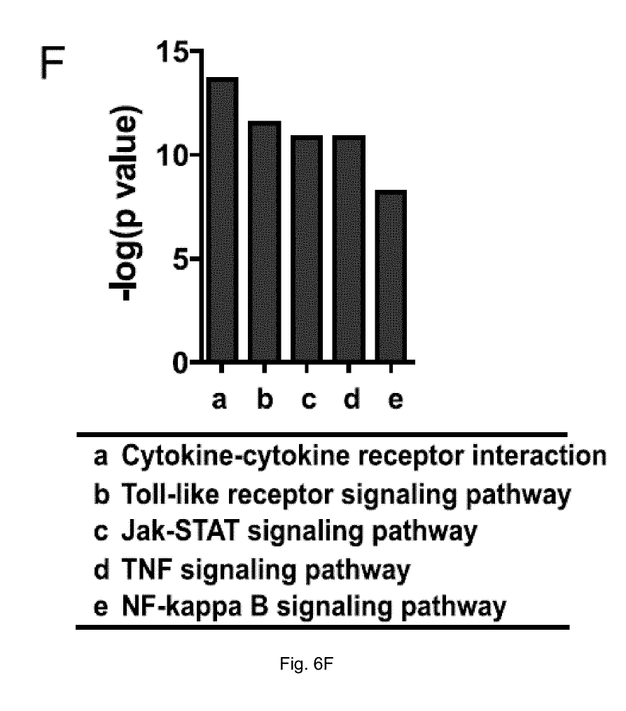

To broadly assess the role of C3ar1/C5ar1 signaling in TLRinduced DC maturation in vivo, we compared the effects of in vivo CpG injection on genes expressed by splenic DCs from WT and C3ar1^{-/-}C5ar1^{-/-} mice using microarrays (FIG. 6). Previous array studies by other investigators performed using in vitro TLR9-stimulated DCs showed that 0.1-10 mg/ml CpG upregulates DC expression of proinflammatory cytokines, type 1 IFNs, and costimulatory signals and that many effects are NF-kB (RelB) dependent (50-52). Other work on murine spleen cell gene expression following in vivo administration of high-dose (400 mg) CpG showed that maximal responses were detected 3-4 h postinjection and resulted in IFN-γ- and TNF-α-initiated inflammatory processes.

Building upon these findings and using our above-identified threshold CpG doses for inducing DC maturation (FIG. 3A), our new analyses show that, at 4 h, 100 μg CpG induces upregulation of ˜1200 WT DC genes (FIG. 6A). These genes mapped to the GO terms immune system process, response to virus, innate immune response, defense response to virus, inflammatory responses, immune response, negative regulation of viral genome replication, cellular responses to interferon-beta, defense response to protozoan, and responses to LPS (data not shown). Included genes are related to the type 1 IFN pathway (IFNb and multiple IRFs), various cytokines and chemokines (e.g., CCL17, CCL22, CCL3, CCL4, CCL5, CXCL1, CXCL10, CXCL11, CXCL13, CXCL3, CXCL9, IL-10, IL-15, IL-27, IL-6, IL12A, IL12B, TNF), signaling pathway genes known to be downstream of TLRs (NFKBIA, CD40, LY96, IKBKE, MYD88, CD80, CD86, MAP3K8, PIK3R5, PIK3R1), and complement components (C3, CFB). Of the 1198 upregulated genes, 608 of them (51%) were induced by TLR9 in DCs from WT mice but not in DCs from CpG-treated C3ar1^{-/-}C5ar1^{-/-} mice (FIG. 6A). These TLR9-induced C3ar1/C5ar1^{-/-} dependent genes mapped to the terms innate immunity, immune systems processes, inflammatory response, and defense response to virus (FIG. 6B, 6D). Although the remaining 590 of 1198 genes (49%) were upregulated in WT and C3ar1^{-/-}C5ar1^{-/-} mice (including cytokine pathway-related genes, TLR pathway-related genes, TNF pathway-related genes, and Jak-STAT pathway-related genes, FIG. 6C, 6E, 6F), greater increases for the majority of them occurred in WT DCs, indicating that C3ar1/C5ar1 signaling amplifies the induction of these other TLR9-induced DC genes. One hundred and fifty-three genes were uniquely upregulated in DCs from CpG-treated C3ar1^{-/-}C5ar1^{-/-} mice. In contrast to the upregulated genes unique to CpG-treated WT DCs, the majority of the upregulated genes in CpG-treated C3ar1^{-/-}C5ar1^{-/-} DCs mapped to noninflammatory pathways, including regulation of myeloid differentiation, regulation of protein kinase cascade, negative regulation of cell differentiation, and regulation of MAPPKKK cascade. Together with the phenotyping and functional data delineated above, the findings support the conclusion that autocrine C3ar1/ C5ar1 signaling is a crucial intermediary required for TLR-induced DC activation.

### BM Cell C3ar1/C5ar1 Signaling is Crucial for TLR9-Induced Cardiac Allograft Rejection

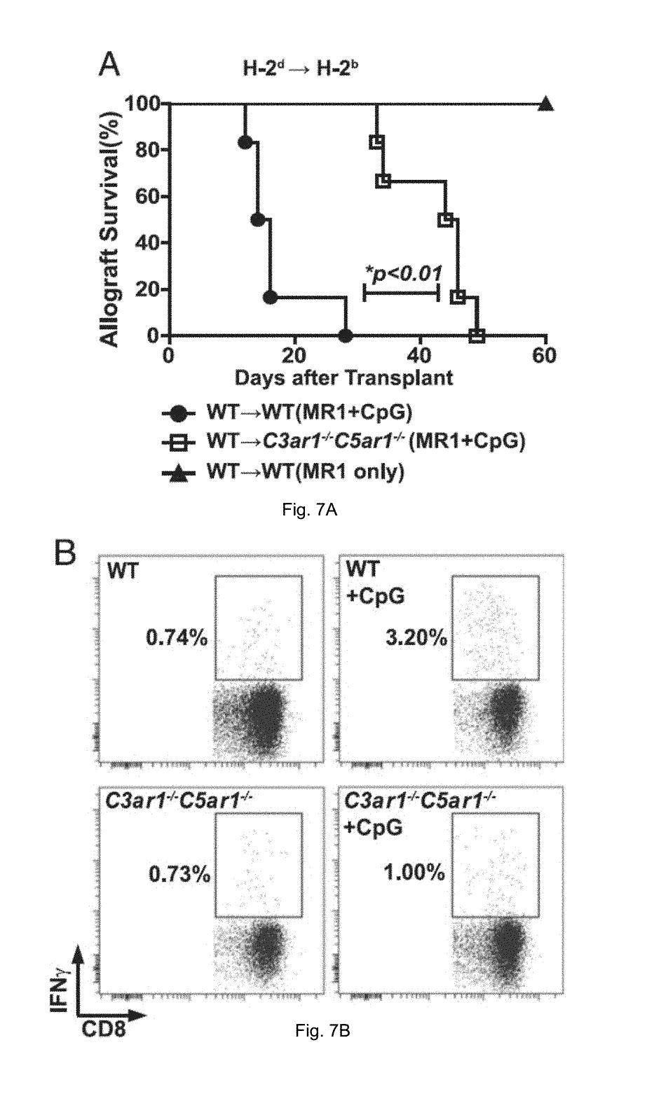

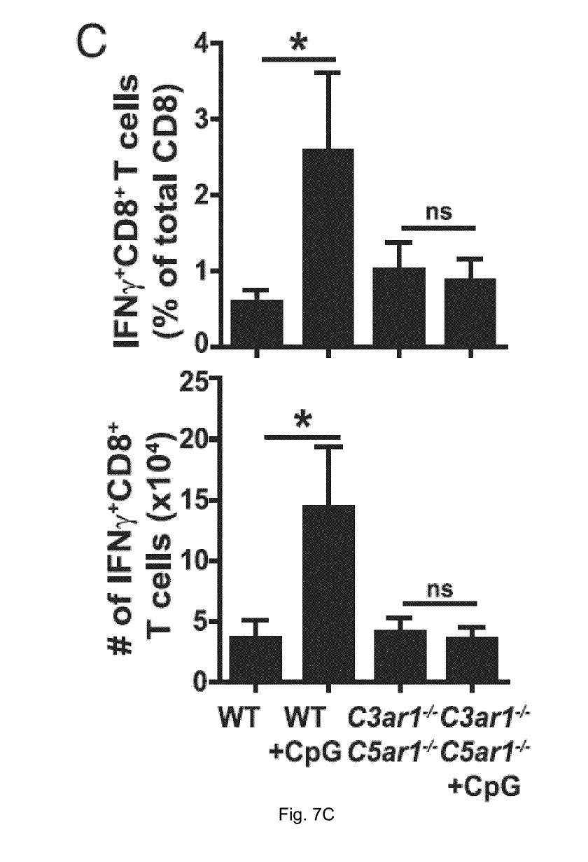

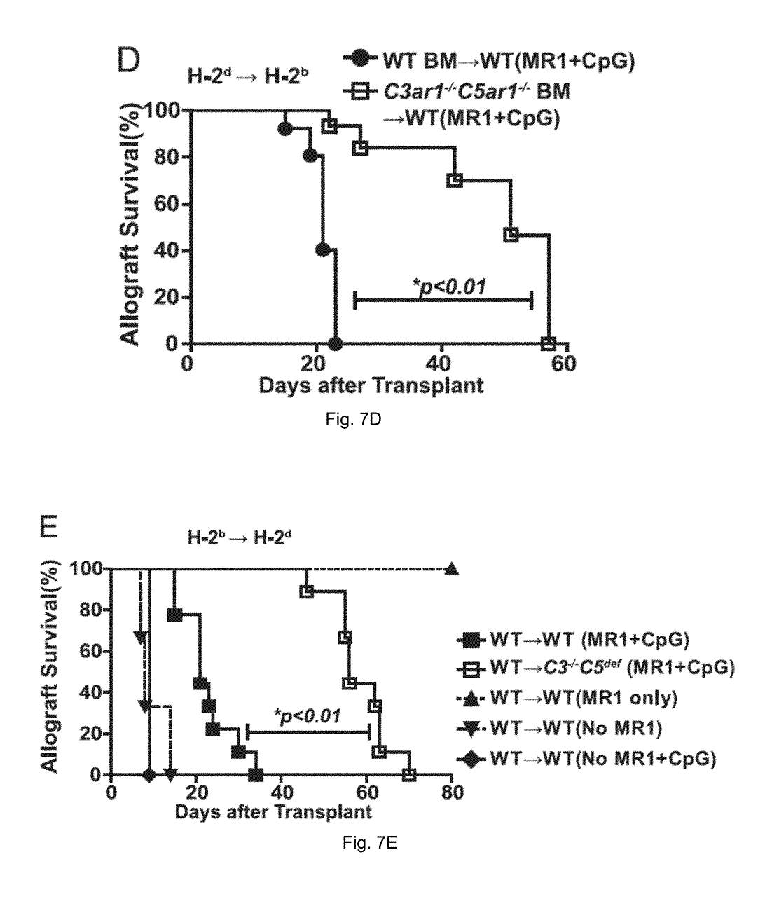

Prior work showed that costimulatory blockade with anti-CD40L mAb (MR1) delays T cellmediated cardiac allograft rejection and can promote T_{reg}-dependent allograft tolerance. CpG administration reverses the effects of MR1, inhibits T_{reg} function, augments alloreactive Teff expansion, and causes rapid transplant rejection. To test the importance of the above-described connection between TLR activation and DC-derived complement activation in a clinically relevant system, we used this transplantation model. We transplanted groups of B6 WT and C3ar1^{-/-}C5ar1^{-/-} recipients with allogeneic BALB/c hearts and treated them with MR1, with or without CpG (FIG. 7). Consistent with previous reports, allografts transplanted into MR1-treated WT recipients survived for >90 d, whereas posttransplant CpG administration caused cessation of graft heartbeat (with histological cellular rejection, data not shown), with a median survival time (MST) of 15 d (p<0.01 versus MR1-treated WT controls). In contrast, allografts transplanted into MR1+CpG-treated C3ar1^{-/-}C5ar1^{-/-} recipients survived longer, with an MST of 45 d (p<0.01 versus MR1+CpG WT controls, FIG. 7A). In the absence of MR1, all WT and C3ar1^{-/-}C5ar1^{-/-} recipients rejected their grafts by day 9 (data not shown).

We repeated the transplant experiments and quantified donorreactive CD8⁺ T cells on day 14 (all allografts beating). Although CpG administration to MR1-treated WT recipients augmented the frequencies and total numbers of splenic donor-reactive IFN-γ-producing CD8⁺ T cells (FIG. 7B, 7C), CpG administration had no effect on the low frequencies or total numbers of donor-reactive T cells detected in the MR1-treated C3ar1^{-/-}C5ar1^{-/-} recipients. No donor-reactive Abs were detected in the sera of any MR1-treated recipients (data not shown).

To test the hypothesis that the prolonged allograft survival of CpG-treated C3ar1^{-/-}C5ar1^{-/-} mice is causally linked to the interconnection of TLR signaling and C3ar1/C5ar1 signaling in immune cells, we performed transplant studies in (CD45.2 H-2^{b}) C3ar1^{-/-}C5ar1^{-/-} BM→(CD45.1) WT chimeras and H-2^{b} WT→WT controls. Ten weeks after documenting ≥90% donor chimerism (data not shown), we transplanted the chimeras with allogeneic hearts and treated them with MR1, with or without CpG (FIG. 7D). Heart grafts transplanted into the chimeras with C3ar1^{-/-}C5ar1^{-/-} BM survived longer than did those transplanted into chimeras with WT BM (MST of 51 and 21 d, respectively, p<0.01). Similar results (FIG. 7E) were obtained in MR1-treated, H-2^{d} C3^{-/-}C5^{def} recipients (MST of 56 d, p<0.01 versus WT control) compared with WT recipients+MR1/CpG (MST of 21 d) and in corresponding BM chimeras (WT BM→WT CpG+MR1 [MST of 34 d] versus C3⁻⁻C5^{def} BM→WT CpG+MR1 [MST of 50 d], p<0.05, n=3-4 per group, data not shown), ascribing the CpG reversal of MR1 costimulatory blockade to heightened immune cell C3ar1/ C5ar1 signaling.

### TLR9-Induced Foxp3 DownRegulation in Tregs is Dependent on C3ar1/C5ar1 Signaling

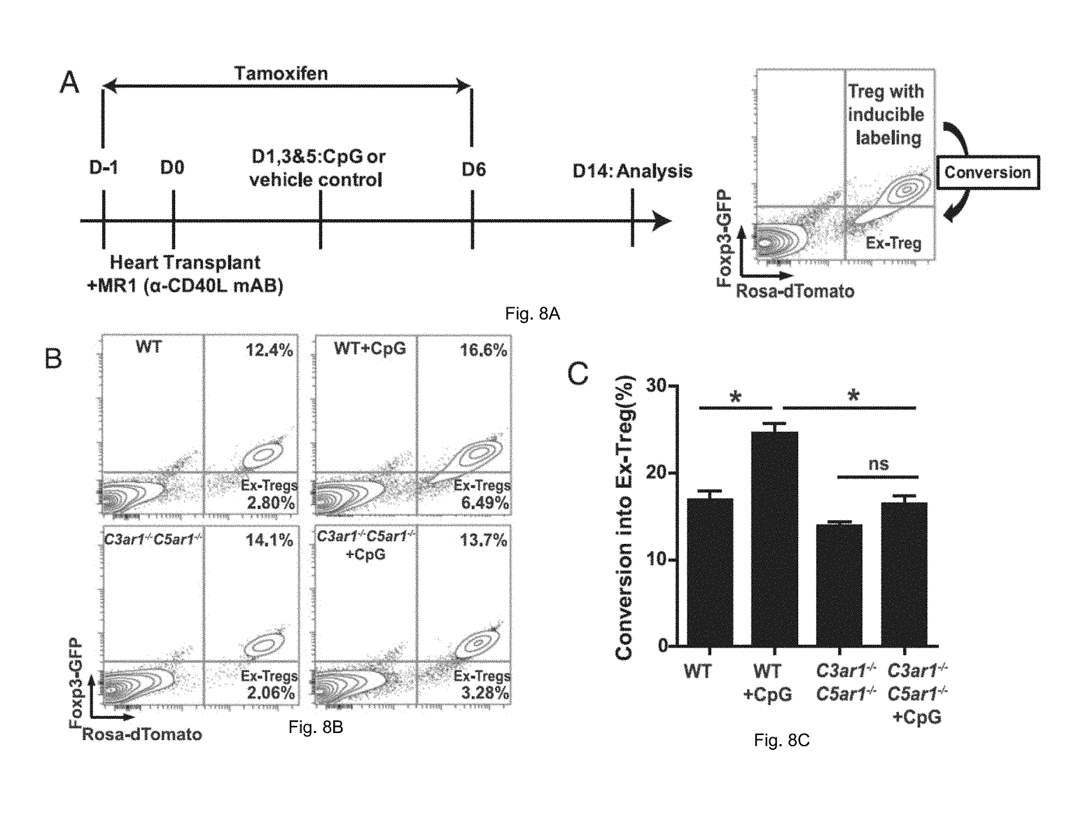

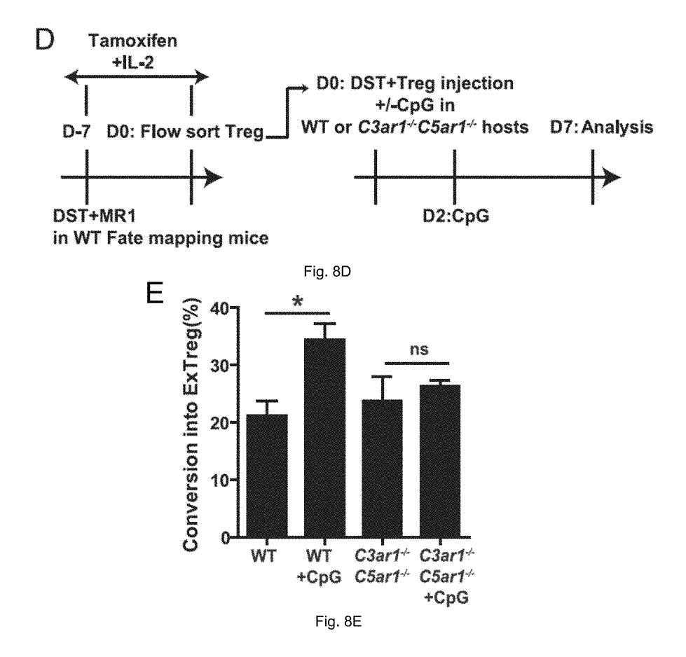

Prior work by other investigators showed that costimulatory blockade with MR1 induces donor-reactive T_{regs}, which prolong cardiac allograft survival/tolerance, and that TLR9 disrupts Foxp3 stability in T_{regs}. In view of these findings, we tested the hypothesis that TLR9 signaling impairs Foxp3 stability through a C3arl/C5ar1-dependent mechanism. We used B6 Rosa (dTomato)XFoxp3CreERT2-GFP fate-mapping mice, in which Foxp3⁺ cells constitutively express GFP, and tamoxifen treatment induces expression of dTomato under the Foxp3 promoter (GFP+dTomato⁺). Thereby, dTomato expression without Foxp3 expression (i.e., GFP^{neg}dTomato⁺phenotype) identifies T cells that were previously Foxp3⁺ but have lost Foxp3 expression (termed ex-T_{regs}) (FIG. 8A).

We transplanted allogeneic BALB/c hearts into groups of B6 WT and C3ar1^{-/-}C5ar1^{-/-}T_{reg} fate-mapping recipient mice.We treated both groups with post-transplant MR1 (to induce T_{regs} and prolong allograft survival) and with tamoxifen to induce Foxp3-GFP+dTomato+T_{regs}. We then administered CpG or vehicle to parallel groups of heart graft recipients and analyzed their spleen cells 14 d post-transplantation (FIG. 8A). CpG administration increased ex-T_{reg} formation from ˜16 to 25% in WT recipients (p<0.05, FIG. 8B, 8C). In contrast, ex-T_{reg} formation in C3ar1^{-/-}C5ar1^{-/-} fate-mapping mice did not increase following CpG treatment (p=not significant).

To test whether T_{reg} extrinsic, systemic C3ar1/C5ar1 signaling mediates in vivo T_{reg} instability following CpG stimulation, we induced dTomato⁺Foxp3-GFP⁺ T_{regs} in WT B6 fate-mapping mice by injecting them with allogeneic BALB/c spleen cells plus MR1 in the presence of tamoxifen and IL-2 (FIG. 8D). Seven days later, we sorted the Foxp3-GFP⁺dTomato⁺WT T_{regs} and adoptively transferred them into WT or C3ar1^{-/-}C5ar1^{-/-} hosts. We then administered BALB/c spleen cells, with or without CpG DNA, to the adoptive hosts and analyzed the splenic T cells on day 14. These assays showed that CpG significantly increased ex-T_{reg} formation in WT adoptive hosts but had no effect on ex-T_{reg} formation in C3ar1^{-/-}C5ar1^{-/-} adoptive recipients (FIG. 8E).

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, We claim:

1. A method of method of inhibiting Toll like receptor (TLR) signaling in dendritic cells (DCs) of a subject in need thereof, the method comprising:
administering at least one complement antagonist to the DCs at an amount effective to substantially inhibits C3a receptor (C3aR) and/or C5a receptor (C5aR) signal transduction in the DCs induced by TLR signaling.

2. The method of claim 1, wherein the TLR signaling is associated with a T cell mediated disorder and/or B cell mediated disorder.

3. The method of claim 2, wherein the T cell mediated disorder and/or B cell mediated disorder is selected from the group consisting of achlorhydra autoimmune active chronic hepatitis, acute disseminated encephalomyelitis, acute hemorrhagic leukoencephalitis, Addison's disease, agammaglobulinemia, alopecia areata, Alzheimer's disease, amyotrophic lateral sclerosis, ankylosing spondylitis, anti-gbm/tbm nephritis, antiphospholipid syndrome, antisynthetase syndrome, aplastic anemia, arthritis, atopic allergy, atopic dermatitis, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune polyendocrine syndrome, autoimmune progesterone dermatitis, autoimmune thrombocytopenia purpura, autoimmune uveitis, balo disease/balo concentric sclerosis, bechets syndrome, Berger's disease, Bickerstaff's encephalitis, blau syndrome, bullous pemphigoid, castleman's disease, chagas disease, chronic fatigue immune dysfunction syndrome, chronic inflammatory demyelinating polyneuropathy, chronic lyme disease, chronic obstructive pulmonary disease, churg-strauss syndrome, cicatricial pemphigoid, coeliac disease, cogan syndrome, cold agglutinin disease, cranial arteritis, crest syndrome, Crohns disease, Cushing's syndrome, Dego's disease, Dercum's disease, dermatitis herpetiformis, dermatomyositis, diabetes mellitus type 1, Dressler's syndrome, discoid lupus erythematosus, eczema, endometriosis, enthesitis-related arthritis, eosinophilic fasciitis, epidermolysis bullosa acquisita, essential mixed cryoglobulinemia, evan's syndrome, fibrodysplasia ossificans progressive, fibromyalgia, fibromyositis, fibrosing aveolitis, gastritis, gastrointestinal pemphigoid, giant cell arteritis, glomerulonephritis, Goodpasture's syndrome, Graves' disease, Guillain-barre syndrome (gbs), Hashimoto's encephalitis, Hashimoto's thyroiditis, henoch-schonlein purpura, hidradenitis suppurativa, Hughes syndrome, inflammatory bowel disease (IBD), idiopathic inflammatory demyelinating diseases, idiopathic pulmonary fibrosis, idiopathic thrombocytopenic purpura, iga nephropathy, inflammatory demyelinating polyneuopathy, interstitial cystitis, irritable bowel syndrome (ibs), Kawasaki's disease, lichen planus, Lou Gehrig's disease, lupoid hepatitis, lupus erythematosus, meniere's disease, microscopic polyangiitis, mixed connective tissue disease, morphea, multiple myeloma, multiple sclerosis, myasthenia gravis, myositis, narcolepsy, neuromyelitis optica, neuromyotonia, occular cicatricial pemphigoid, opsoclonus myoclonus syndrome, ord thyroiditis, Parkinson's disease, pars planitis, pemphigus, pemphigus vulgaris, pernicious anaemia, polymyalgia rheumatic, polymyositis, primary biliary cirrhosis, primary sclerosing cholangitis, progressive inflammatory neuropathy, psoriasis, psoriatic arthritis, raynaud phenomenon, relapsing polychondritis, Reiter's syndrome, rheumatoid arthritis, rheumatoid fever, sarcoidosis, schizophrenia, Schmidt syndrome, Schnitzler syndrome, scleritis, scleroderma, Sjogren's syndrome, spondyloarthropathy, sticky blood syndrome, still's disease, stiff person syndrome, sydenham chorea, sweet syndrome, takayasu's arteritis, temporal arteritis, transverse myelitis, ulcerative colitis, undifferentiated connective tissue disease, undifferentiated spondyloarthropathy, vasculitis, vitiligo, Wegener's granulomatosis, Wilson's syndrome, Wiskott-Aldrich syndrome, hypersensitivity reactions of the skin, atherosclerosis, ischemia-reperfusion injury, myocardial infarction, and restenosis.

4. The method of claim 1, wherein the at least one complement antagonist substantially inhibits interaction of at least one of C3a or C5a with respectively the C3aR or C5aR of the DCs.

5. The method of claim 4, the at least one complement antagonist being selected from the group consisting of a small molecule, a polypeptide, and a polynucleotide.

6. The method of claim 5, the polypeptide comprising an antibody directed against at least one of C3, C5, C3 convertase, C5 convertase, C3a, C5a, C3aR, or C5aR.

7. The method of claim 1, the step of administering the at least one complement antagonist including administering to the DCs a C3a antagonist and a C5a antagonist and/or a C3aR antagonist and a C5aR antagonist.

8. The method of claim 1, wherein the complement antagonist substantially inhibits dendritic cell C5a/C3a production and T cell C5aR/C3aR signal transduction in the subject.

9. A method of treating a T cell mediated disorder in a subject, the method comprising:
administering to the subject a therapeutically effective amount of at least one complement antagonist and a pharmaceutically acceptable carrier, wherein the at least one complement antagonist substantially inhibits interaction of at least one of C3a or C5a with the C3a receptors (C3aR) and C5a receptors (C5aR) on interacting dendritic cells and T cells in the subject.

10. The method of claim 9, wherein the complement antagonist does not substantially systemic complement activation.

11. The method of claim 9, wherein the T cell mediated disorder is selected from the group consisting of achlorhydra autoimmune active chronic hepatitis, acute disseminated encephalomyelitis, acute hemorrhagic leukoencephalitis, Addison's disease, agammaglobulinemia, alopecia areata, Alzheimer's disease, amyotrophic lateral sclerosis, ankylosing spondylitis, anti-gbm/tbm nephritis, antiphospholipid syndrome, antisynthetase syndrome, aplastic anemia, arthritis, atopic allergy, atopic dermatitis, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune polyendocrine syndrome, autoimmune progesterone dermatitis, autoimmune thrombocytopenia purpura, autoimmune uveitis, balo disease/balo concentric sclerosis, bechets syndrome, Berger's disease, Bickerstaff's encephalitis, blau syndrome, bullous pemphigoid, castleman's disease, chagas disease, chronic fatigue immune dysfunction syndrome, chronic inflammatory demyelinating polyneuropathy, chronic lyme disease, chronic obstructive pulmonary disease, churg-strauss syndrome, cicatricial pemphigoid, coeliac disease, cogan syndrome, cold agglutinin disease, cranial arteritis, crest syndrome, Crohns disease, Cushing's syndrome, Dego's disease, Dercum's disease, dermatitis herpetiformis, dermatomyositis, diabetes mellitus type 1, Dressler's syndrome, discoid lupus erythematosus, eczema, endometriosis, enthesitis-related arthritis, eosinophilic fasciitis, epidermolysis bullosa acquisita, essential mixed cryoglobulinemia, evan's syndrome, fibrodysplasia ossificans progressive, fibromyalgia, fibromyositis, fibrosing aveolitis, gastritis, gastrointestinal pemphigoid, giant cell arteritis, glomerulonephritis, Goodpasture's syndrome, Graves' disease, Guillain-barre syndrome (gbs), Hashimoto's encephalitis, Hashimoto's thyroiditis, henoch-schonlein purpura, hidradenitis suppurativa, Hughes syndrome, inflammatory bowel disease (IBD), idiopathic inflammatory demyelinating diseases, idiopathic pulmonary fibrosis, idiopathic thrombocytopenic purpura, iga nephropathy, inflammatory demyelinating polyneuopathy, interstitial cystitis, irritable bowel syndrome (ibs), Kawasaki's disease, lichen planus, Lou Gehrig's disease, lupoid hepatitis, lupus erythematosus, meniere's disease, microscopic polyangiitis, mixed connective tissue disease, morphea, multiple myeloma, multiple sclerosis, myasthenia gravis, myositis, narcolepsy, neuromyelitis optica, neuromyotonia, occular cicatricial pemphigoid, opsoclonus myoclonus syndrome, ord thyroiditis, Parkinson's disease, pars planitis, pemphigus, pemphigus vulgaris, pernicious anaemia, polymyalgia rheumatic, polymyositis, primary biliary cirrhosis, primary sclerosing cholangitis, progressive inflammatory neuropathy, psoriasis, psoriatic arthritis, raynaud phenomenon, relapsing polychondritis, Reiter's syndrome, rheumatoid arthritis, rheumatoid fever, sarcoidosis, schizophrenia, Schmidt syndrome, Schnitzler syndrome, scleritis, scleroderma, Sjogren's syndrome, spondyloarthropathy, sticky blood syndrome, still's disease, stiff person syndrome, sydenham chorea, sweet syndrome, takayasu's arteritis, temporal arteritis, transverse myelitis, ulcerative colitis, undifferentiated connective tissue disease, undifferentiated spondyloarthropathy, vasculitis, vitiligo, Wegener's granulomatosis, Wilson's syndrome, Wiskott-Aldrich syndrome, hypersensitivity reactions of the skin, atherosclerosis, ischemia-reperfusion injury, myocardial infarction, and restenosis.

12. The method of claim 9, the at least one complement antagonist being selected from the group consisting of a small molecule, a polypeptide, and a polynucleotide.

13. The method of claim 12, the polypeptide comprising an antibody directed against at least one of C3, C5, C3 convertase, C5 convertase, C3a, C5a, C3aR, or C5aR.

14. The method of claim 9, the step of administering the at least one complement antagonist including administering to the subject an antibody directed against C5aR and an antibody directed against C3aR.

15. The method of claim 9, the step of administering the at least one complement antagonist including administering to the subject an antibody directed against C5a and an antibody directed against C3a.

16. The method of claim 9, the complement antagonist and a pharmaceutically acceptable carrier being administered locally to a site of T cell mediated disorder in the subject.

17. The method of claim 9, the complement antagonist being conjugated to a targeting moiety that targets a site of T cell mediated disorder being treated.

18. The method of claim 9, the complement antagonist and a pharmaceutically acceptable carrier being administered systemically to the subject being treated.

19. The method of claim 9, wherein the complement antagonist inhibits dendritic cell C5a/C3a production and T cell C5aR/C3aR signal transduction in the subject.
